SEQUENCE LISTING (1 sequence)

SEQ ID NO: 1 - protein, 15 aa, synthetic construct
GLNDIFEAQKIEWHE

(19) United States
(12) Patent Application Publication
(10) Pub. No.: US 2025/0064762 A1
(43) Pub. Date: Feb. 27, 2025

(54) A SUBSTITUTED AROMATIC DICARBONIC ACID AMIDE AS AN INHIBITOR OF THE FERROPTOSIS SUPPRESSOR PROTEIN-1 (FSP1)

(71) Applicant: JULIUS-MAXIMILIANS-UNIVERSITÄT WÜRZBURG, Würzburg (DE)

(72) Inventor: Antje GOHLA, Würzburg (DE)

(21) Appl. No.: 18/724,152

(22) PCT Filed: Dec. 23, 2022
(86) PCT No.: PCT/EP2022/087694

§ 371 Date: Jun. 25, 2024

(30) Foreign Application Priority Data
Dec. 27, 2021 (EP) 21217834.7

(51) Int. Cl. A61K 31/167; A61P 35/00

(52) U.S. Cl. CPC A61K 31/167; A61P 35/00

(57) ABSTRACT

The invention refers to a substituted aromatic dicarbonic acid amide compound according to formula I for use in the treatment of cancer, in particular human and/or rodent cancer. Furthermore, the present invention relates to a pharmaceutical composition comprising a compound according to formula I for use in the treatment of cancer and to the use of a compound of formula I for inducing phospholipid dependent ferroptosis in an in vitro cell sample.

### FIELD OF THE INVENTION

The invention refers to a substituted aromatic dicarbonic acid amide compound according to formula I for use in the treatment of cancer, in particular human and/or rodent cancer. Furthermore, the present invention relates to a pharmaceutical composition comprising a compound according to formula I for use in the treatment of cancer and to the use of a compound of formula I for inducing phospholipid dependent ferroptosis in an in vitro cell sample.

### BACKGROUND OF THE INVENTION

Ferroptosis is a non-apoptotic, regulated form of cell death driven by iron-dependent phospholipid peroxidation. Therapy-resistant cancer cells, in particular those in the mesenchymal, metastasis-prone state, are highly vulnerable to ferroptosis. Ferroptosis thus represents a targetable susceptibility in certain cancers, and the concept of pharmacological ferroptosis induction emerges as a potential antitumor strategy.

Ferroptosis relies on iron, reactive oxygen species and the availability of phospholipids with polyunsaturated fatty acids (see FIG. 1A). Because cellular metabolism (in particular lipid, amino acid, iron and selenium metabolism), redox homeostasis, nutrients, inputs from extra- and intracellular signaling and environmental stressors can all feed into the induction and execution of ferroptosis, this form of cell death is under the perpetual control of various cell-intrinsic ferroptosis suppressing pathways. The canonical anti-ferroptotic axis involves cysteine-uptake via the cysteine/glutamate antiporter (system x_{c}⁻), and glutathione (GSH) biosynthesis. GSH is the most abundant reductant in mammalian cells and a cofactor of the major cellular ferroptosis suppressor glutathione peroxidase-4 (GPX4), which detoxifies phospholipid hydroperoxides to their corresponding phospholipid alcohols (see FIG. 1A). Recently, the ferroptosis suppressor protein (FSP1)-ubiquinone axis has been identified as a potent GPX4-independent ferroptosis surveillance pathway. FSP1 (gene name: AIFM2) inhibits lipid peroxidation and ferroptosis by reducing ubiquinone to ubiquinol, and/or by regenerating the oxidized α-tocopheryl radical to α-tocopherol (vitamin E), the most powerful natural chain-breaking lipid anti-oxidant (see FIG. 1A). Alternate protective pathways may involve di/tetrahydrobiopterin- or squalene-mediated inhibition of deleterious lipid peroxidation. Although a number of experimental system x_{c}⁻ and GPX4 inhibitors have been developed, only one small molecule inhibitor of FSP1 (iFSP1) has been described so far (Doll et al., Nature 2019, DOI: 10.1038/s41586-019-1707-0). Enzymatic activity assays using recombinant, highly purified FSP1 proteins in vitro demonstrate that iFSP1 is an inhibitor of human FSP1, but that it is much less active against murine FSP1 (see also Examples 7-10 herein). While iFSP1 is able to inhibit endogenous and overexpressed FSP1 in human cancer cell lines, and thereby sensitize these cells to ferroptotic death, iFSP1 is completely ineffective in mouse or rat cancer cell lines (see FIG. 20A). Indeed, iFSP1 concentrations >10 μM are non-specifically toxic to cells (see FIGS. 21A and 21B). iFSP1 can therefore not be used to investigate the effects of FSP1 inhibition in rodent cells.

Genetically defined rodent cell lines, organotypic in vitro cultures of mouse or rat organ slices, organoids/spheroids/assembloids and in vivo murine tumor models are indispensable for the development and testing of novel cancer treatment strategies. Importantly, the inclusion of rodent models is also a compulsory part of the pre-clinical test phase of potential medicines. The unique value of rodent and in particular murine cell lines and animal models in pre-clinical cancer research lies in the possibility to recapitulate the complex genetic and epigenetic aberrations that are found in patients' tumors. Using techniques such as Cre/loxP-mediated gene knock-out or knock-in, RNA interference-induced gene knockdown, and/or CRISPR/Cas-based genome editing, oncogenic driver mutations and/or losses of tumor suppressors can be combinatorically modeled in an intact and immune-competent organism.

While human cancer cell lines xenografted into mice have been essential for in vivo cancer research, these models are intrinsically unsuitable for the development of immune-oncological therapies, because they require transplantation into immunodeficient animals. Similarly, patient-derived xenografts established in immunocompromised mice do not allow for the assessment of adaptive anti-cancer immunity mechanisms. To circumvent these limitations, considerable efforts are directed towards the generation of “humanized mice”, in which human immune cells or precursors are engrafted into immunodeficient mouse strains. Nevertheless, immune responses in these models are severely limited.

Taken together, from a pharmacological and drug development point of view, it is in general highly advantageous to have compounds in hand that are active against both, human and rodent proteins (or other target structures).

In the field of ferroptosis and cancer, there is currently an unmet need to identify novel FSP1 inhibitors that are applicable not only in human, but also in rodent cell lines. Such compounds can be tested, further refined and validated in genetically defined, immune-competent mouse tumor models for the evaluation of conventional, targeted, and immune therapies for cancer.

In view of the above, compounds capable of modulating FSP1 are useful for treating cancer. Most cancers, for example, melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma are likely to be sensitive to ferroptosis. Accordingly, there is a need for compounds that are active against FSP1, in particular against both human and rodent FSP1.

### Objectives and Summary of the Invention

It is therefore an object of the present invention to provide compounds which modulate FSP1, in particular, compounds which inhibit FSP1 activity for use in the treatment of cancer. It is yet another object of the present invention to provide a pharmaceutical composition comprising said compounds for use in the treatment of cancer.

The above objects can be achieved by the compounds according to formula I as defined herein as well as pharmaceutical compositions comprising said compounds according to formula I for use in the treatment of cancer.

Thus, the invention is based on the surprising finding that the compounds according to formula I as defined herein modulate FSP1, in particular inhibit FSP1. Accordingly, said compounds according to formula I as defined herein can be used for the treatment of cancer, in particular for the treatment of ferroptosis-sensitive cancers.

Hence, a first aspect of the present invention relates to a compound according to formula I

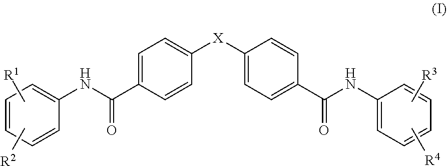

or a salt, tautomer, stereoisomer or N-oxide thereof,
wherein
X is O, S or CH₂;
R¹, R³ are independently of each other selected from H, halogen, CN, NO₂, C₁-C₄-alkyl, C₁-C₄-haloalkyl, C₂-C₆-alkenyl, C₂-C₆-alkynyl, OR^{a}, SR^{a}, C(═O)OR^{a}, NR^{b}R^{c}, and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};

R², R⁴ are independently of each other selected from H, halogen, CN, NO₂, C₁-C₄-alkyl, C₁-C₄-haloalkyl, C₂-C₆-alkenyl, C₂-C₆-alkynyl, OR^{a}, SR^{a}, NR^{b}R^{c}; and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
and wherein
R^{a} is H, C₁-C₄-alkyl, C₁-C₄-haloalkyl, or C₂-C₆-alkenyl;
R^{b}, R^{c} are independently of each other selected from H, C₁-C₄-alkyl, and C₂-C₆-alkenyl;
R^{x} is halogen, CN, NO₂, C₁-C₄-alkyl, or C₁-C₄-haloalkyl,
for use in the treatment of cancer.

In one embodiment of the present invention, in said compound of formula I for use
X is O;
R¹, R³ are independently of each other selected from H, halogen and C(═O)OR^{a};
R², R⁴ are independently of each other selected from H, halogen, CN, NO₂, and C₁-C₄-alkyl;
R^{a} is H, C₁-C₄-alkyl.

In another embodiment of the present invention, in said compound of formula I for use
X is O;
R¹, R³ are C(═O)OR^{a};
R², R⁴ are H,
R^{a} is H, or C₁-C₄-alkyl.

In yet another embodiment of the present invention, the compound of formula I for said use is a compound according to the following formula

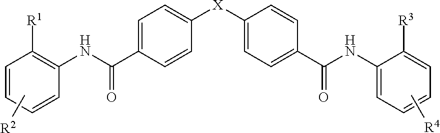

or a pharmaceutically acceptable salt thereof,
wherein
X is O;
R¹, R³ are C(═O)OR^{a};
R², R⁴ are H,
R^{a} is H, or C₁-C₄-alkyl.

In a more preferred embodiment of the present invention, the compound of formula I for said use is a compound according to the following formula

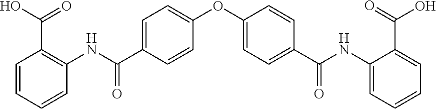 [SMILES: O=C(Nc1ccccc1C(=O)O)c1ccc(Oc2ccc(C(=O)Nc3ccccc3C(=O)O)cc2)cc1]

or a pharmaceutically acceptable salt thereof.

In another embodiment of the present invention, the compound as defined above is for use in the treatment of mammalian cancers, preferably human and/or rodent cancers.

In one embodiment of the present invention, the compound as defined above is for use in the treatment of mesenchymal cancer and/or metastasis-prone cancer.

In one embodiment of the present invention, the compound as defined above is for use in the treatment of persister cancer cells.

In another embodiment of the present invention, the compound as defined above is for use in the treatment of ferroptosis-sensitive cancer.

In another embodiment of the present invention, the compound as defined above is for use in the treatment of FSP1-dependent cancer cells.

In another embodiment of the present invention, the compound as defined above is for use in the treatment of GPX4-deficient cancer cells.

In yet another embodiment of the present invention, the compound as defined above is for use in the treatment of solid cancer.

In yet another embodiment of the present invention, the compound as defined above is for use in the treatment of liquid cancer.

In a preferred embodiment of the present invention, the compound as defined above is for use in the treatment of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

In another aspect, the present invention refers to the use of a compound as defined above for the manufacture of a medicament for the treatment and/or prophylaxis of cancer.

In one embodiment of said use, the compound as defined above is for the manufacture of a medicament for the treatment and/or prophylaxis of mesenchymal cancer and/or metastasis-prone cancer.

In another embodiment of said use, the compound as defined above is for the manufacture of a medicament for the treatment and/or prophylaxis of persister cancer cells.

In another embodiment of said use, the compound as defined above is for the manufacture of a medicament for the treatment and/or prophylaxis of ferroptosis-sensitive cancer.

In another embodiment of said use, the compound as defined above is for the manufacture of a medicament for the treatment and/or prophylaxis of FSP1-dependent cancer cells.

In another embodiment of said use, the compound as defined above is for the manufacture of a medicament for the treatment and/or prophylaxis of GPX4-deficient cancer cells.

In yet another embodiment of said use, the compound as defined above is for the manufacture of a medicament for the treatment and/or prophylaxis of solid cancer.

In yet another embodiment of said use, the compound as defined above is for the manufacture of a medicament for the treatment and/or prophylaxis of liquid cancer.

In another embodiment of said use, the compound as defined above is for the manufacture of a medicament for the treatment and/or prophylaxis of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

In another aspect, the present invention refers to a pharmaceutical composition comprising the compound as defined above together with a pharmaceutically acceptable carrier and optionally further therapeutic excipient for use in the treatment of cancer, preferably wherein the further therapeutic excipient is a chemotherapeutic agent.

In one embodiment, the present invention refers to a pharmaceutical composition as defined above, for use in the treatment of mesenchymal cancer and/or metastasis-prone cancer.

In one embodiment, the present invention refers to a pharmaceutical composition as defined above, for use in the treatment of persister cancer cells.

In another embodiment, the present invention refers to a pharmaceutical composition as defined above, for use in the treatment of ferroptosis-sensitive cancer.

In one embodiment, the present invention refers to a pharmaceutical composition as defined above, for use in the treatment of FSP1-dependent cancer cells.

In another embodiment, the present invention refers to a pharmaceutical composition as defined above, for use in the treatment of GPX4-deficient cancer cells.

In yet another embodiment, the present invention refers to a pharmaceutical composition as defined above, for use in the treatment of solid cancer.

In yet another embodiment, the present invention refers to a pharmaceutical composition as defined above, for use in the treatment of liquid cancer.

In another embodiment, the present invention refers to a pharmaceutical composition as defined above, for use in the treatment of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

In a further aspect, the present invention relates to the use of a compound or a pharmaceutically acceptable salt thereof as defined above for inducing phospholipid dependent ferroptosis in an in vitro cell sample.

In one embodiment of said use, the compound as defined above acts as a modulator of FSP1. In one particular embodiment of said use, the compound as defined above acts as an antagonist of FSP1.

In one embodiment, the cell sample is a tumor cell line, an organ slice, an organoid, a spheroid or an assembloid.

### BRIEF DESCRIPTION OF THE FIGURES

FIG. 1A: Left, Schematic of ferroptosis, a regulated form of cell death driven by iron-dependent phospholipid peroxidation. ROS, reactive oxygen species; PUFA-PL, phospholipids with polyunsaturated fatty acids. Many cancer cells (including metastatic, drug-resistant persister cells) show increased susceptibility to ferroptosis, and ferroptosis induction may be explored as an antitumor strategy.

Right, Ferroptosis-suppressing pathways. The canonical anti-ferroptotic axis involves cystine-uptake via the cystine/glutamate antiporter (system xc-), and glutathione (GSH) biosynthesis. GSH is the most abundant reductant in mammalian cells and a cofactor of the glutathione peroxidase-4 (GPX4), which catalyzes the reduction of phospholipid hydroperoxides (PL-OOH) to their corresponding phospholipid alcohols (PL-OH). Oxidized glutathione (GSSG) is recycled by glutathione disulfide reductase (GSR) using electrons provided by NADPH (B). The ferroptosis suppressor protein (FSP1)-ubiquinone axis (A) completely protects cells against ferroptosis induced by pharmacological GPX4 inhibition (using small molecule compounds such as RSL3 or FIN56) or by genetic GPX4 deletion. GPX4 activity can also be inhibited by inhibiting cystine import using erastin, or by inhibiting glutamate-cysteine ligase (GCL), the rate-limiting enzyme of GSH biosynthesis, using buthionine sulfoximine (BSO). FSP1 inhibits lipid peroxidation and ferroptosis by reducing ubiquinone to ubiquinol, and/or by regenerating the oxidized α-tocopheryl radical to α-tocopherol (vitamin E), the most powerful natural chain-breaking anti-oxidant in lipids. The activity of human FSP1 can be inhibited by the small molecule inhibitor iFSP1. Alternate pathways involving squalen- or di/tetrahydrobiopterin-mediated inhibition of lipid peroxidation are not shown. (Figure modified and legend adapted from Jiang, Stockwell and Conrad, Nature Reviews Molecular Biology 2021; doi: 10.1038/s41580-020-00324-8).

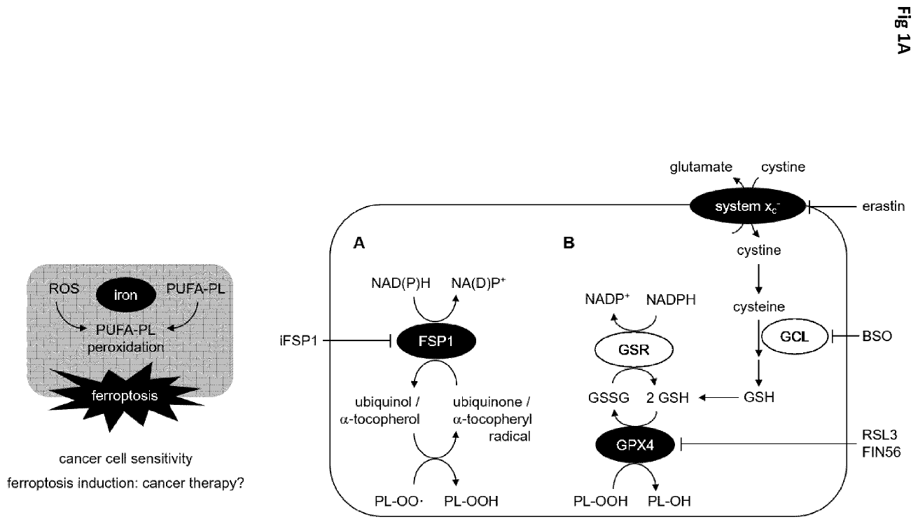

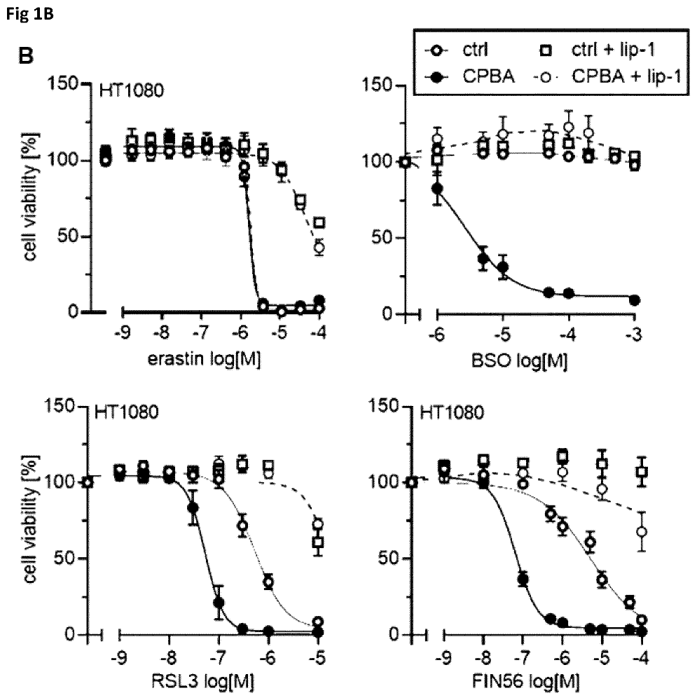

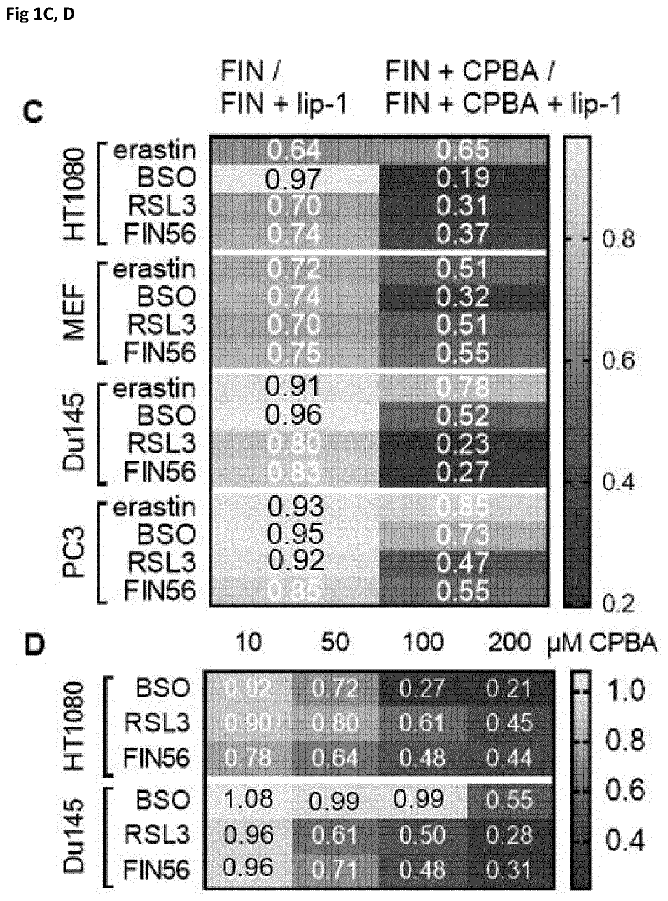

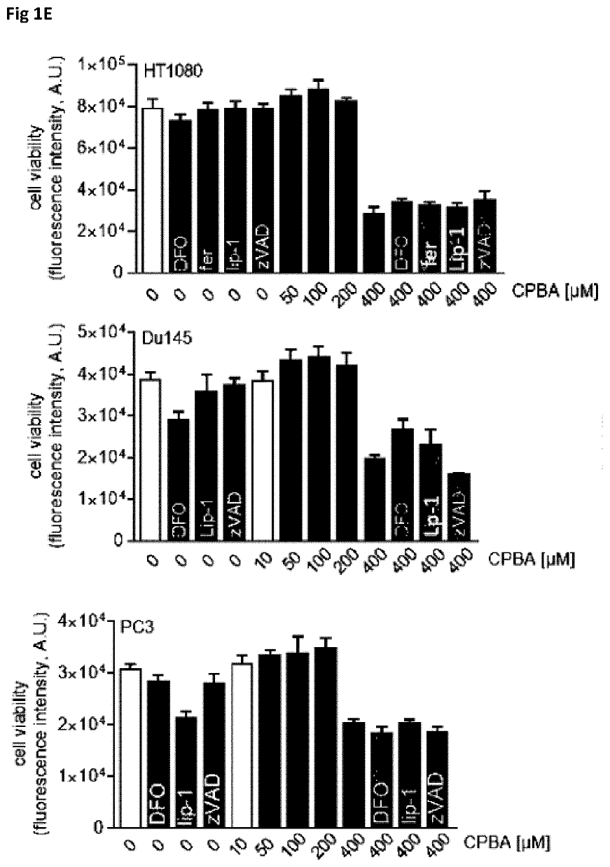

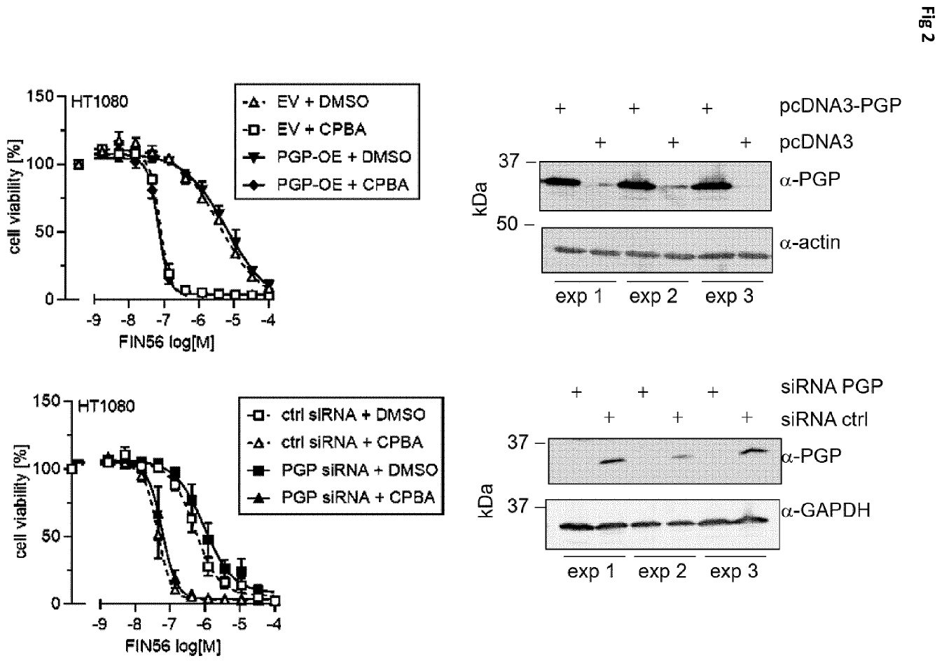

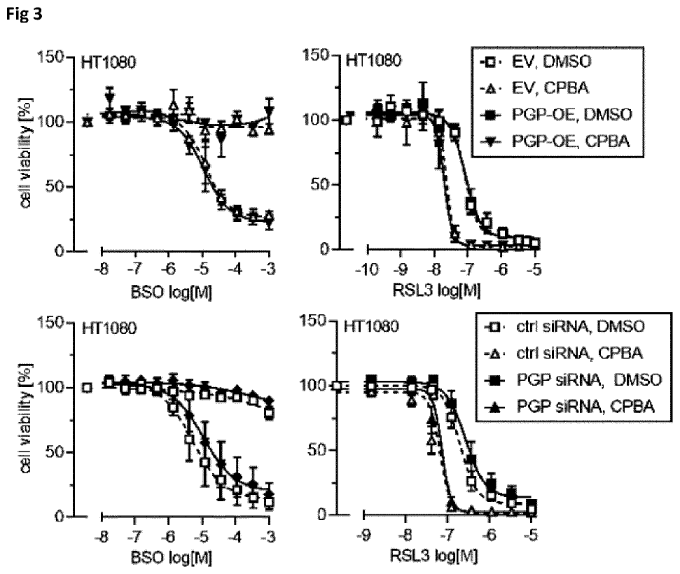

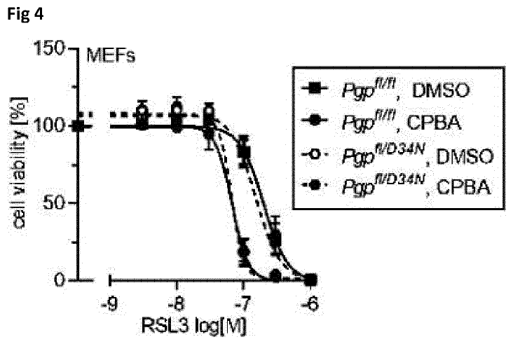

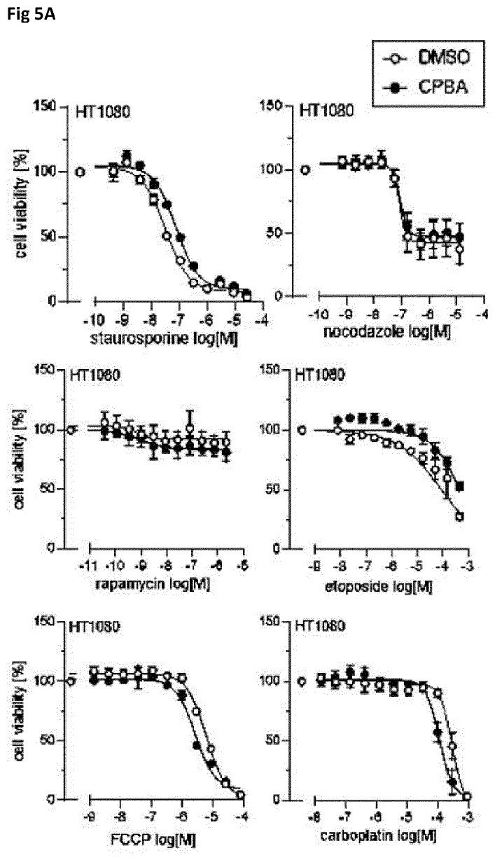

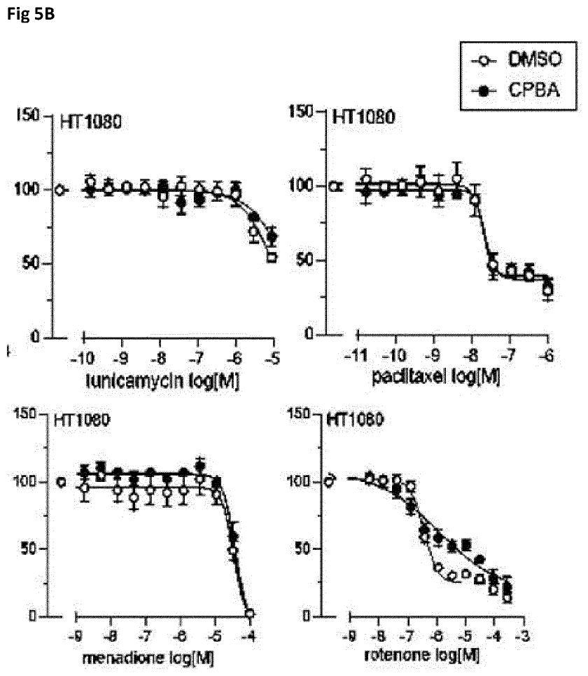

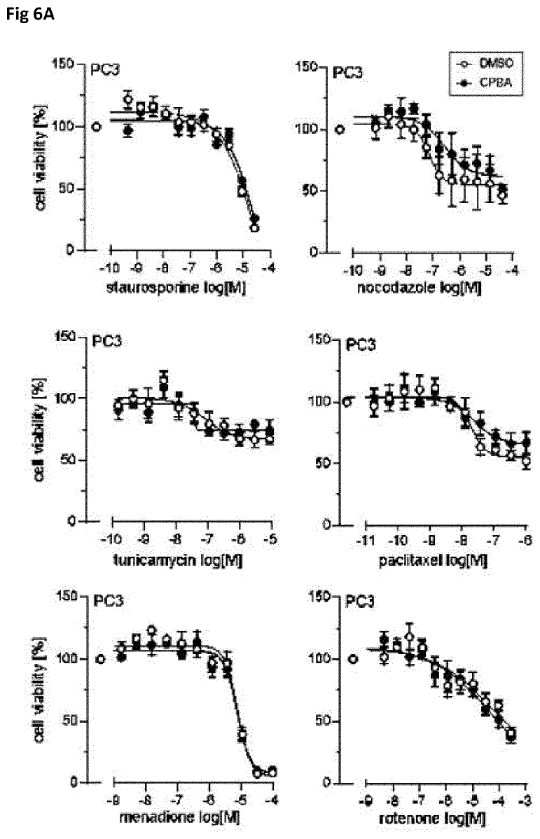

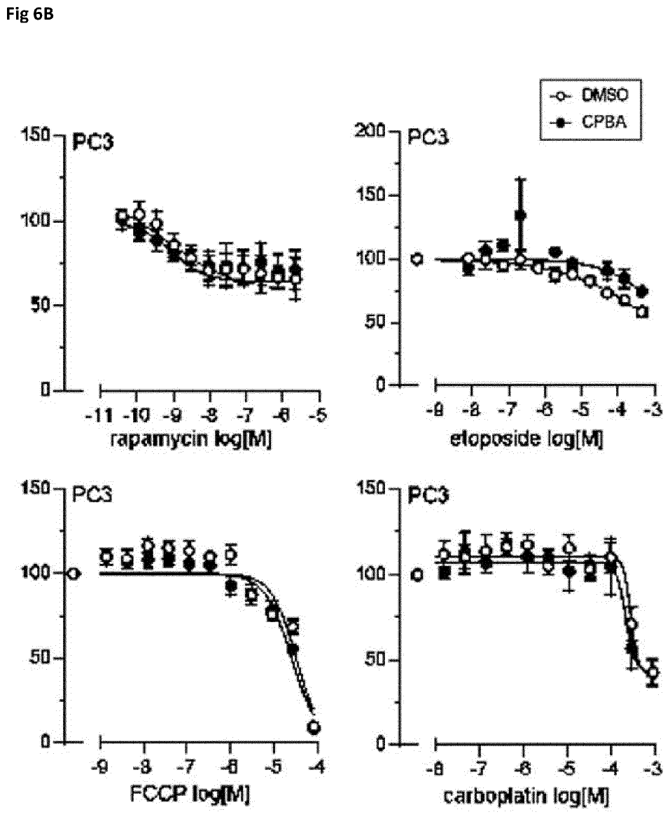

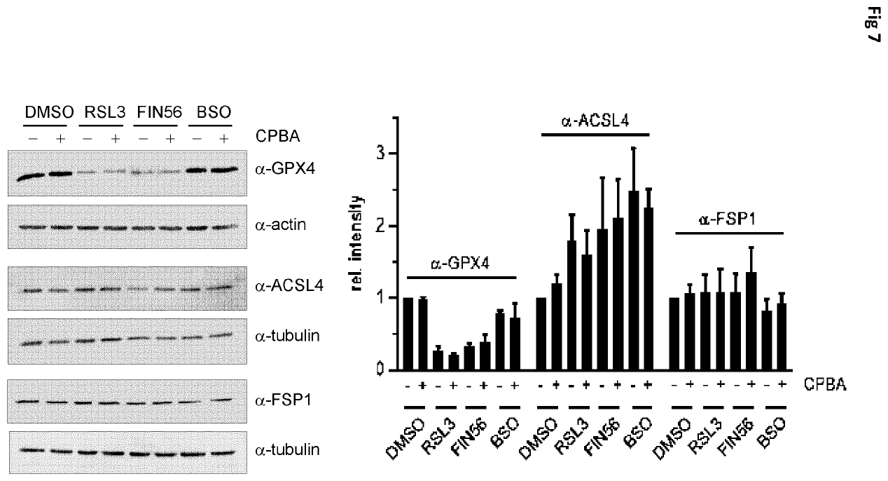

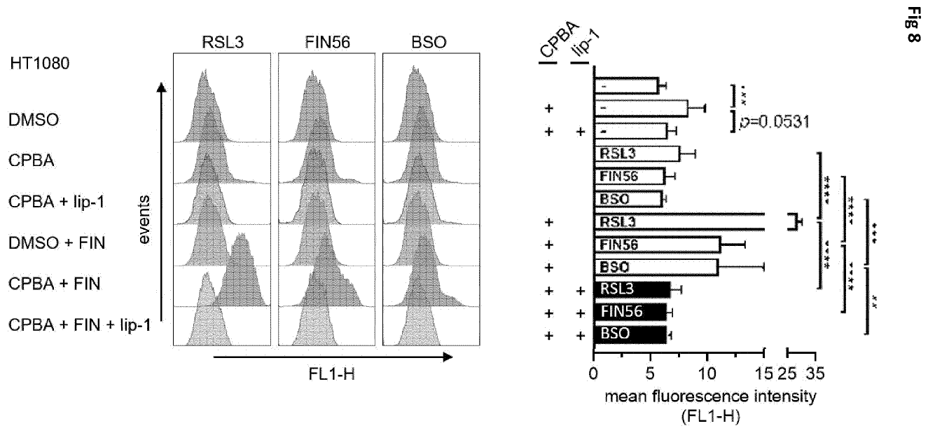

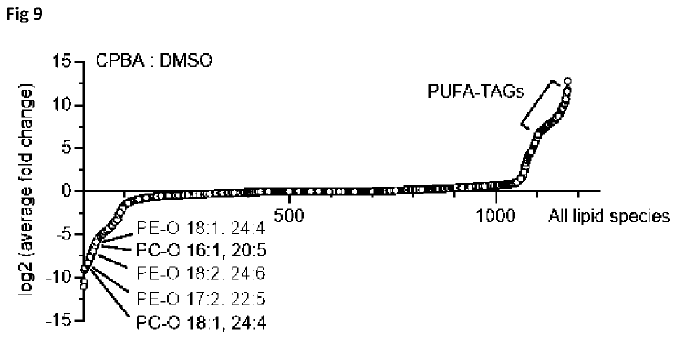

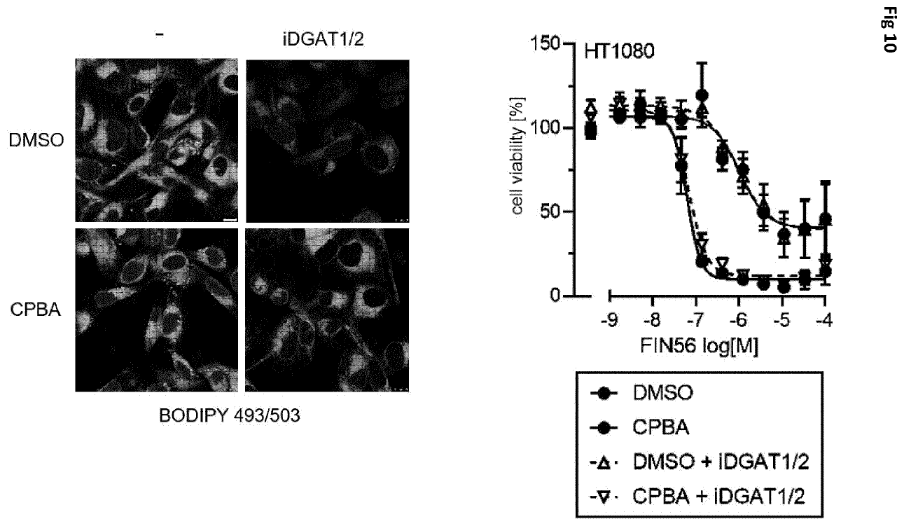

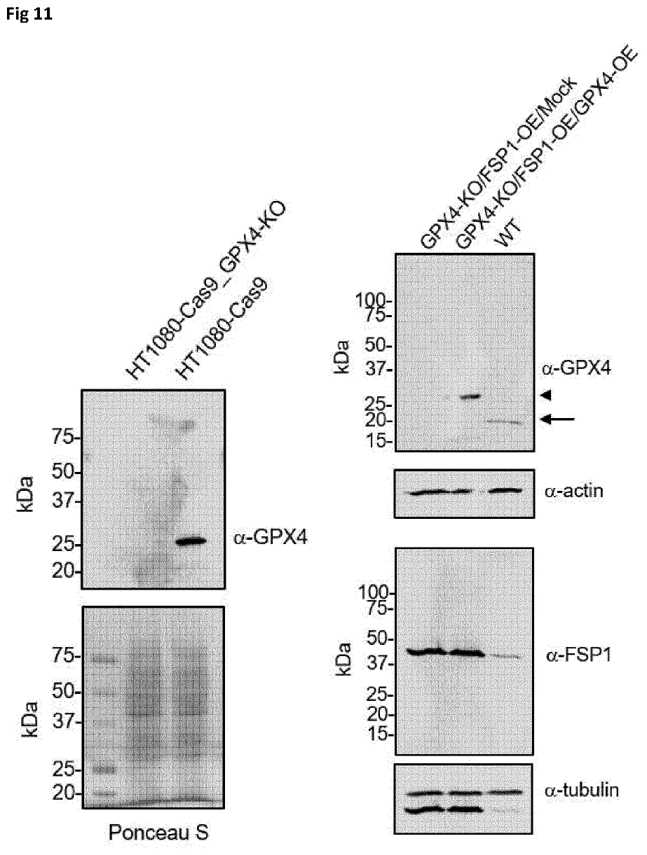

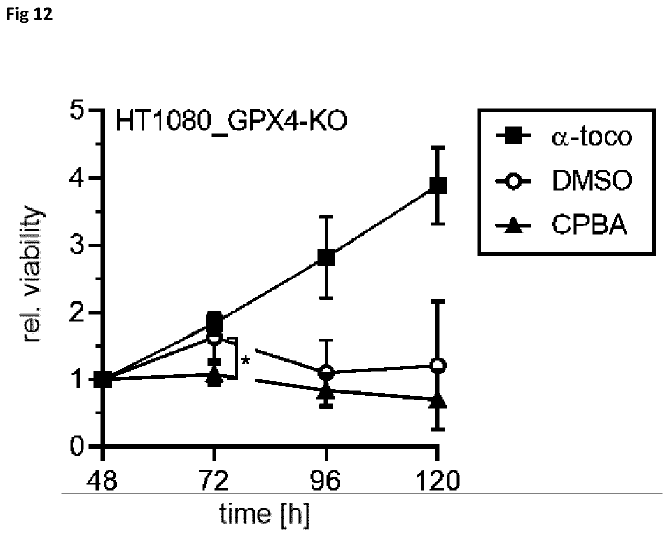

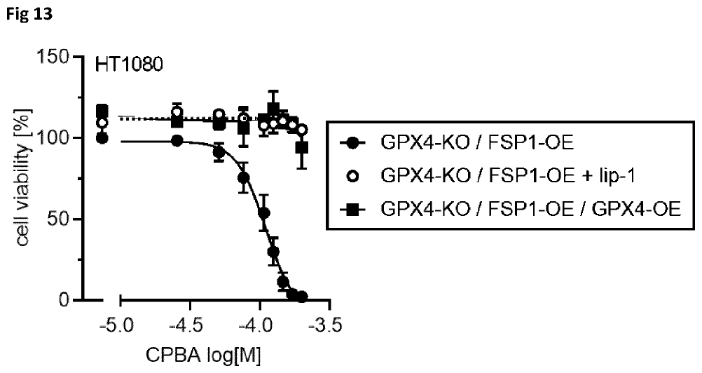

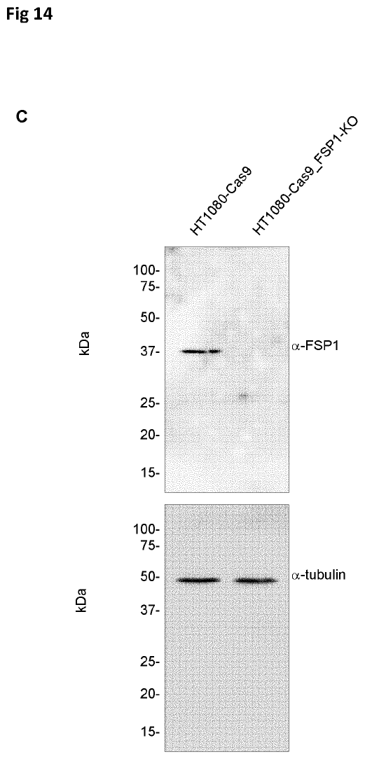

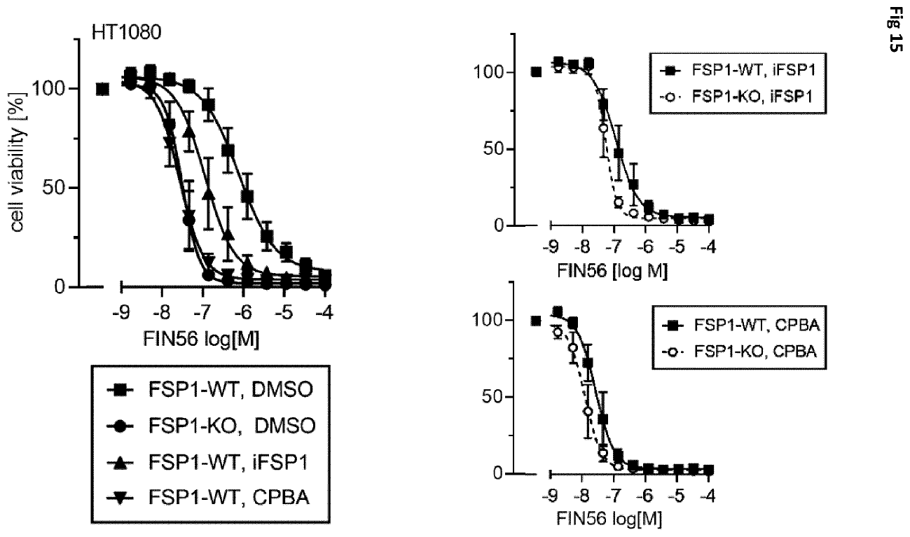

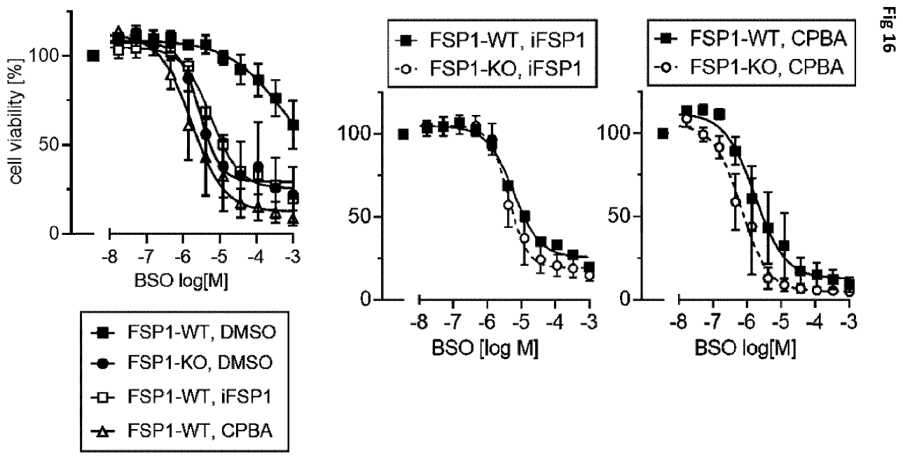

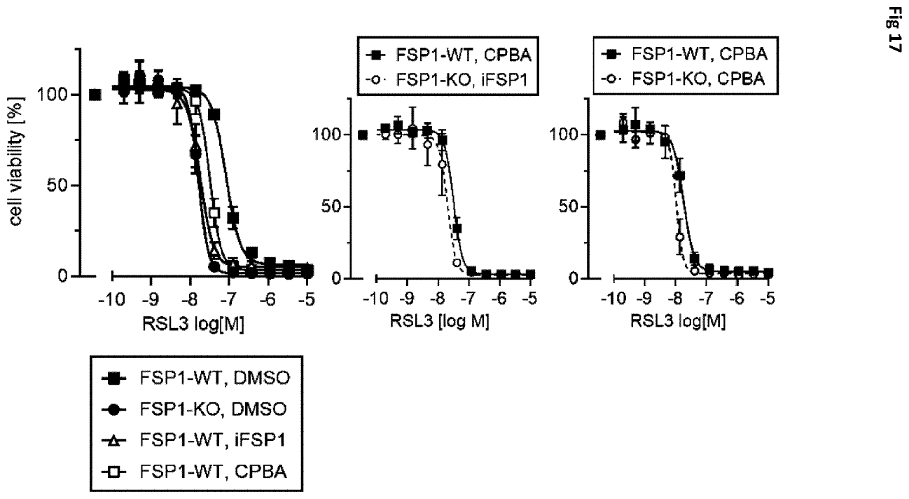

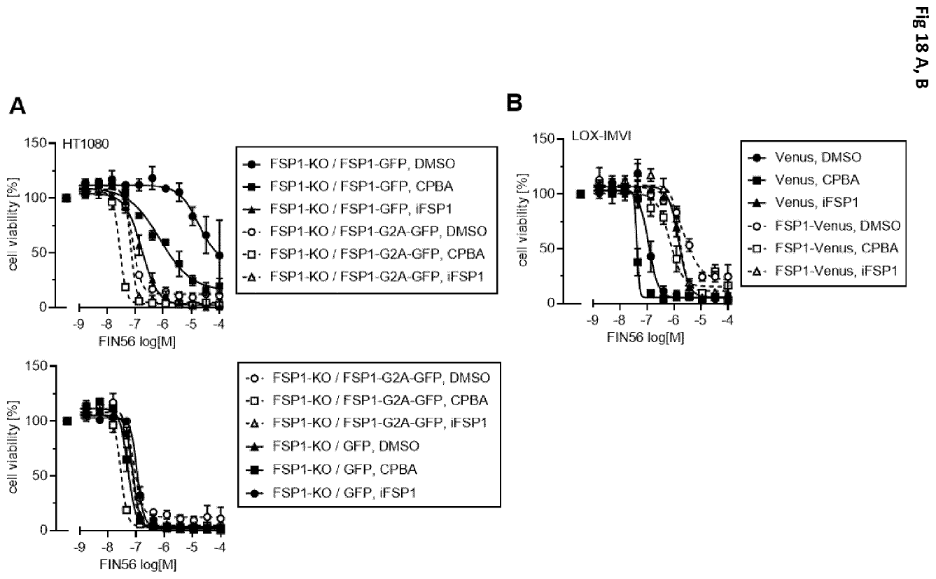

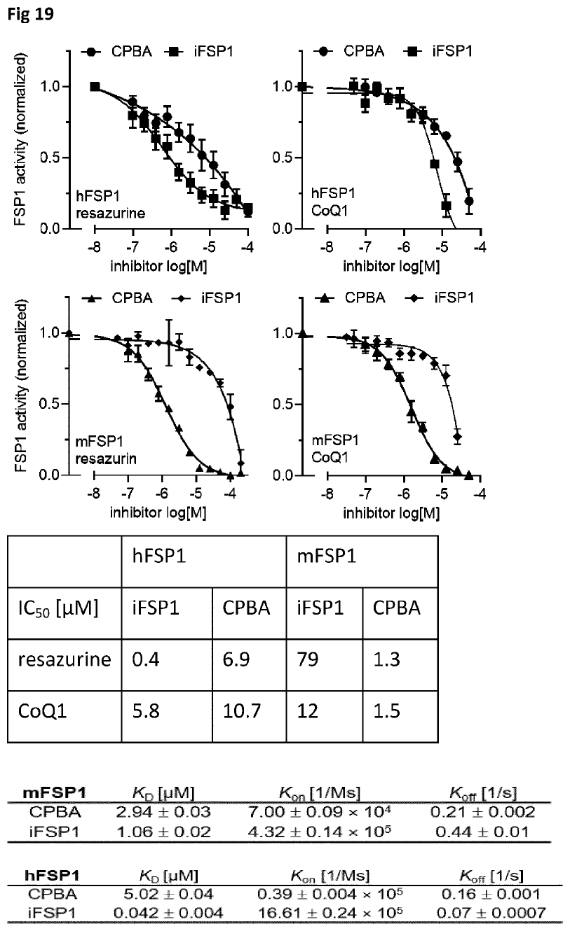

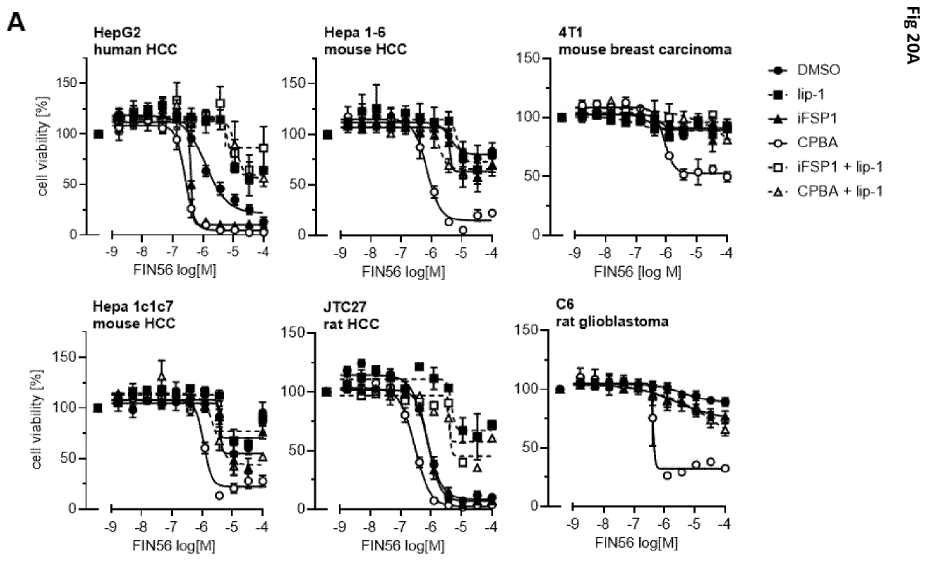

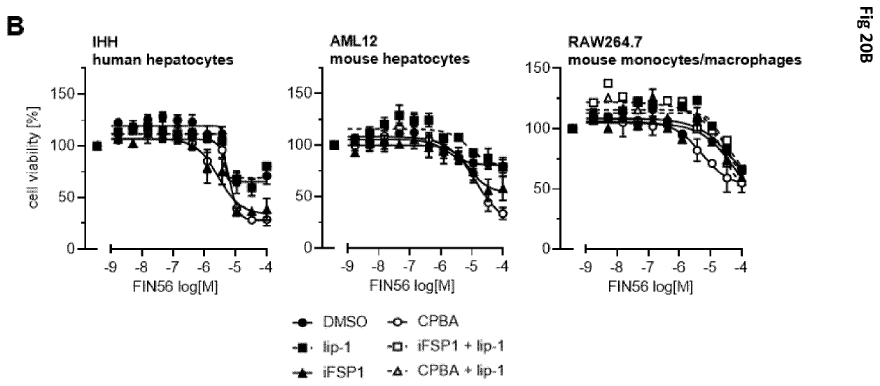

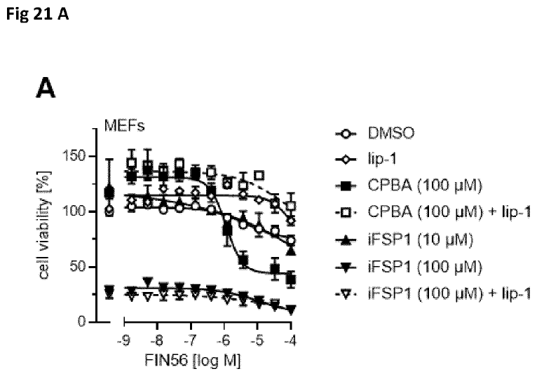

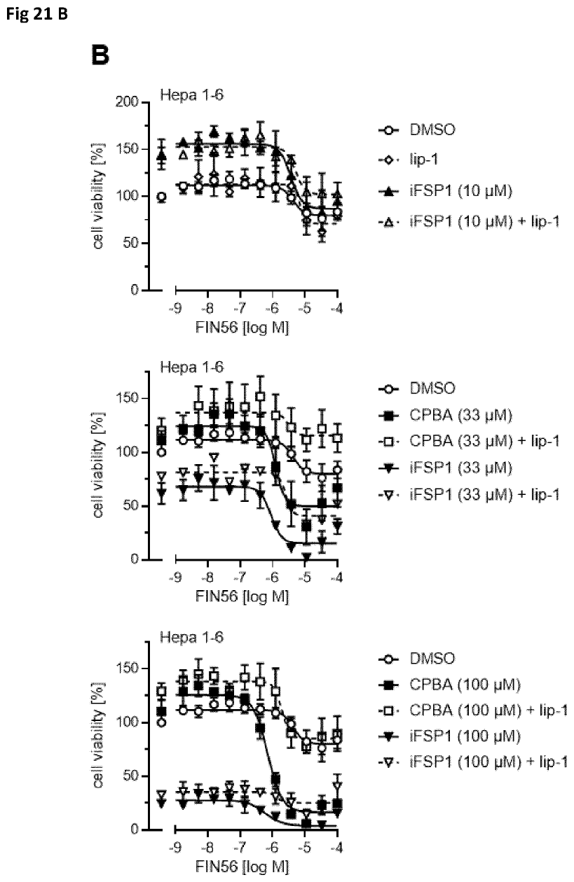

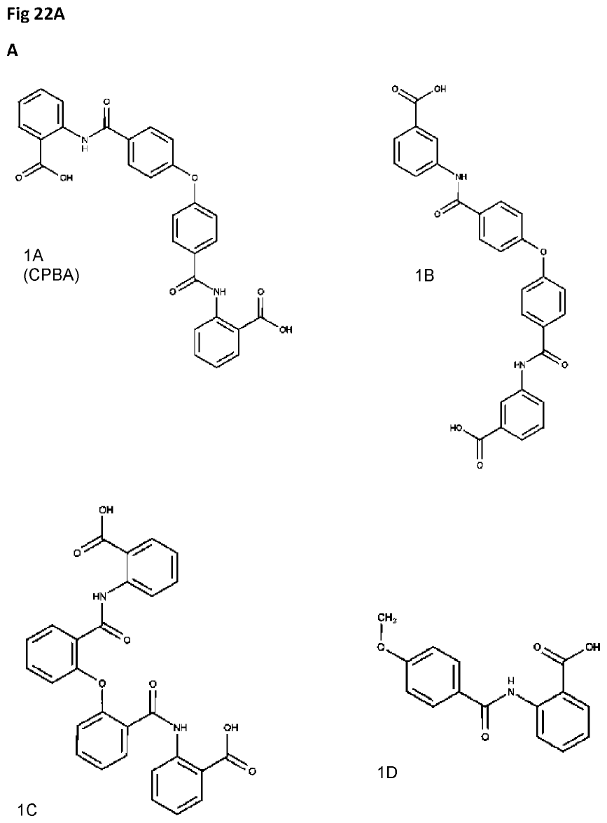

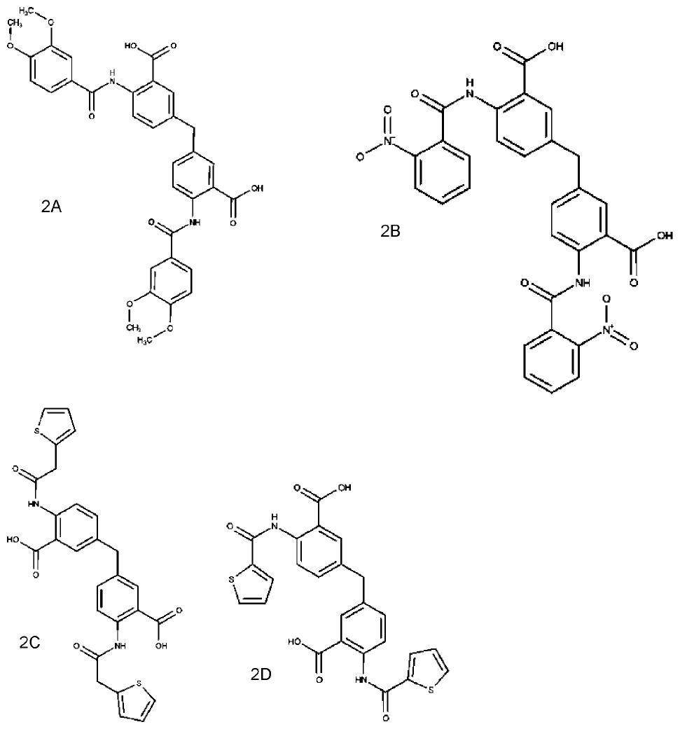

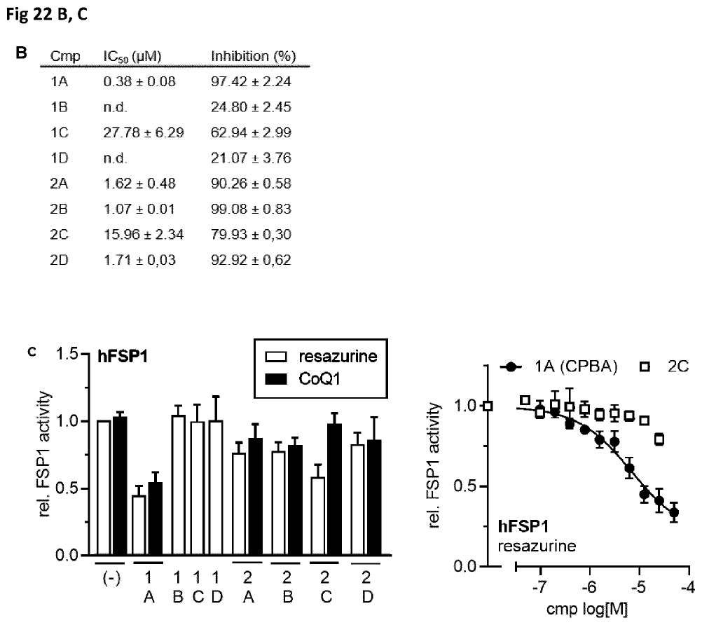

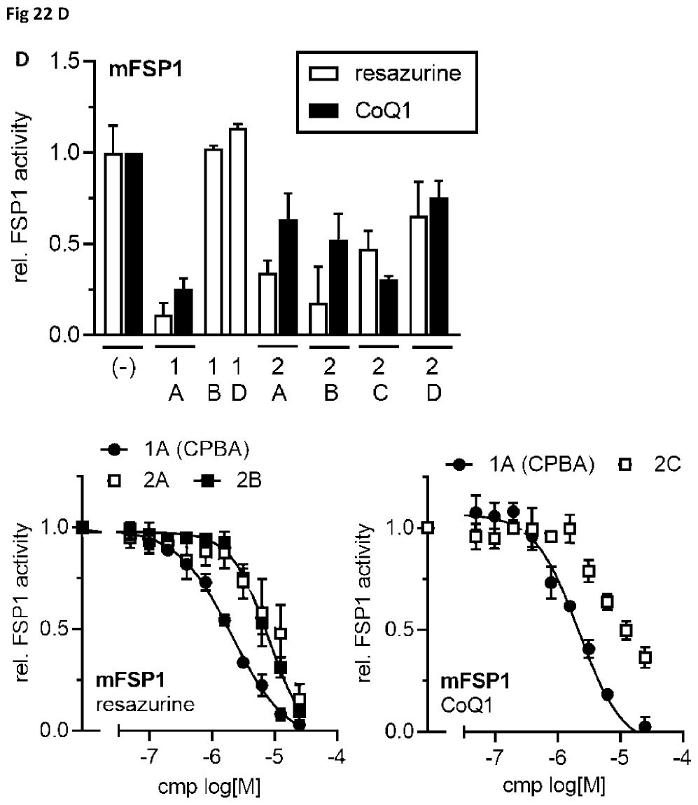

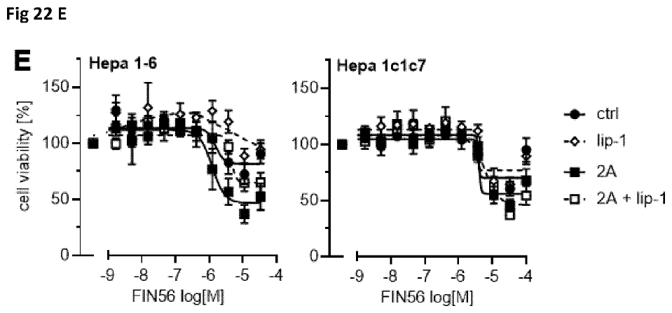

FIG. 1B: HT1080 human fibrosarcoma cells were incubated with serial dilutions of the ferroptosis inducers (FINs) erastin, BSO, RSL3 or FIN56 in the absence or presence of CPBA (100 μM) and the ferroptosis inhibitor liproxstatin-1 (lip-1, 100 nM). Cell viability was analyzed using Aquabluer after 16 h.

FIG. 1C: Area-under-the-curve (AUC) metrics of cell line sensitivity to CPBA in the presence of different FINs. Scoring was based on AUC values, because they reflect changes in IC50 (potency) as well as in efficacy (% inhibition). HT1080 cells, mouse embryonic fibroblasts (MEFs, non-transformed, non-immortalized) and human prostate carcinoma cell lines Du145 and PC3 were incubated with serial dilutions of FINs±CPBA±liproxstatin-1 (HT1080, Du145, PC3: 200 μM CPBA, MEF: 100 μM CPBA; lip-1: 100 nM), and cell viability was analyzed after 16 h. Concentration-response curves were constructed by non-linear regression, and AUC values were calculated (AUC FIN+ctrl/AUC FIN+ctrl+lip-1 and AUC FIN+CPBA/AUC FIN+CPBA+lip-1). The heatplot depicts the concentration-dependent synthetic lethality of CPBA when combined with FINs. The lower the value, the stronger the CPBA-induced effect on FIN-induced ferroptosis.

FIG. 1D: Ferroptosis-sensitization by CPBA is concentration-dependent. HT1080 and PC3 cells were incubated with serial dilutions of FINs+CPBA, and cell viability was analyzed after 16 h. AUC values (AUC FIN+CPBA/AUC FIN+ctrl) were calculated as in FIG. 1C.

FIG. 1E: Effect of increasing CPBA concentrations in the absence of ferroptosis inducers on the viability of HT1080, Du145 or PC3 cells. Cells were incubated with the indicated concentrations of CPBA, and cell viability was analyzed after 16 h. CPBA alone is not cytotoxic at concentrations of up to 200 μM. Non-ferroptotic, non-apoptotic cell death is observed in the presence of 400 μM CPBA. DFO, deferoxamine (an iron-chelator and ferroptosis inhibitor); fer, ferrostatin (a ferroptosis inhibitor); lip-1, liproxstatin-1 (a ferroptosis inhibitor); zVAD, zVAD-FMK (a pan-caspase and apoptosis inhibitor).

FIG. 1 B-E: All data are mean values±S.E.M. of n≥3 independent experiments. Error bars not shown are hidden by the symbols. Ctrl, DMSO solvent control; lip-1, liproxstatin-1.

FIG. 2: Ferroptosis-sensitization by CPBA is independent of PGP. HT1080 cells transiently overexpressing PGP (top), or depleted of PGP using RNA interference (bottom) were treated with CPBA or solvent control as in FIG. 1B. PGP expression levels are documented in the Western Blot in the respective right-hand panels.

All data are mean values ±S.E.M. of n≥3 independent experiments. Error bars not shown are hidden by the symbols. Ctrl, DMSO solvent control; lip-1, liproxstatin-1.

FIG. 3: Ferroptosis-sensitization by CPBA is PGP-independent. HT1080 cells transiently overexpressing PGP (top), or depleted of PGP using RNA interference (bottom) were incubated with serial dilutions of BSO or RSL3, and treated with the DMSO solvent control or CPBA.

FIG. 4: MEFs generated from Pgp-wildtype (Pgp^{fl/fl}) or heterozygous Pgp-inactivated mouse embryos (Pgp^{fl/D34N}) were incubated with serial dilutions of RSL3, and treated with the DMSO solvent control or CPBA. Homozygously Pgp-inactivated MEFs (Pgp^{D34N/D34N}) do not survive under normoxic tissue culture conditions (Segerer et al., Sci Rep 2016; doi: 10.1038/srep35160), and were not investigated in this study. Heterozygous genetic inactivation of Pgp does not recapitulate the ferroptosis-sensitizing effect of CPBA. All data are mean values±S.E.M. of n≥3 independent experiments. Error bars not shown are hidden by the symbols.

FIG. 5A, B, FIG. 6A, B: HT1080 fibrosarcoma (FIG. 5) or PC3 prostate carcinoma cells (FIG. 6) were incubated for 16 h with a panel of cytotoxic drugs (STS, staurosporine; NOC, nocodazole; TUN, tunicamycin; PAX, paclitaxel; ETO, etoposide; MEN, menadione; ROT, rotenone; FCCP, carbonyl cyanide 4-(trifluoromethoxy)phenyl-hydrazone; CPT, carboplatin; 11 serial 1:3 dilutions each), in the absence (DMSO solvent control) or presence of CPBA (100 μM), and cell viability was analyzed. All data are mean values±S.E.M. of n≥3 independent experiments.

FIG. 7: Left panel, HT1080 cells were cultured in the presence of liproxstatin-1 (100 nM), and incubated for 16 h with the solvent control DMSO (0.1%), RSL3 (1 μM), FIN56 (10 μM), or BSO (100 μM) in the presence of either 0.1% DMSO (−) or 100 μM CPBA (+). Cells were lysed and probed with the indicated antibodies. Comparable protein loading was assessed by reprobing the same blots with actin- or tubulin-directed antibodies. Shown are representative blots of n=3 independent experiments performed with different passages (8, 12 and 15) of HT1080 cells. GPX4, glutathione peroxidase-4; FSP1 ferroptosis suppressor protein-1; ACSL4, acyl-CoA synthetase long chain family member 4 (ACSL4).

Right panel, Results summary of densitometric analyses of n=3 experiments performed as shown in the left panel.

FIG. 8: Effects of CPBA on cellular lipid peroxidation were investigated using a BODIPY 581/591-C11 probe. HT1080 cells were incubated for 16 h with DMSO (0.1%, solvent control), CPBA (100 μM), or liproxstatin-1 (lip-1, 100 nM) in the absence or presence of subeffective concentrations of RSL3 (50 nM), FIN56 (50 nM), or BSO (1 μM). Fluorescence intensity was quantified by flow cytometry. Left panel, representative of n=4 (RSL3/FIN56) or n=3 (BSO) independently performed experiments. Right panel, results summary. Statistically significant differences were assessed using one-way ANOVA and Tukey's multiple comparisons test. ****, p<0.0001; ***, p<0.001; **, p<0.01.

FIG. 9: Lipidomic signature of HT1080 cells treated with CPBA (200 μM, 16 h). The plot shows the average of the log 2-transformed fold changes in all detected lipid species, measured in n=4 independently performed experiments. CPBA-treated cells downregulate polyunsaturated fatty acid (PUFA) ether lipids (PE-O, phosphatidylethanolamine ether; PC—O, phosphatidylcholine ether) and upregulate PUFA-triacylglycerides (PUFA-TAGS). This lipidomic signature has recently been described in cancer cells that are initially dependent on GPX4 to evade ferroptosis, and indicates a sensitization to ferroptosis.

FIG. 10: Lipid droplets are dispensable for CPBA-induced sensitization to ferroptosis. Left panel, HT1080 cells were incubated for 16 h with the DMSO solvent control or CPBA (100 μM) in the absence or presence of inhibitors of DGAT1 (T863, IDGAT1; 10 μM) and DGAT2 (PF-06424439, iDGAT2; 5 μM). Cells were fixed, lipid droplets were stained with BODIPY 493/503, and cells were imaged by fluorescence microscopy. Images are representative of >20 imaged fields in n=3 independent experiments. Scale bar, 10 μm. Right panel, HT1080 cells were incubated as described in the left panel, and the effect of CPBA on FIN56-induced ferroptosis in the absence or presence of DGAT1/2 inhibitors and thus lipid droplets was analyzed using Aquabluer. Data are mean values±S.E.M. of n=3 independently performed experiments.

FIG. 11: Western blots of cells lines employed. Note that the reconstituted GPX4 (in GPX4-KO/FSP1-OE/GPX4-OE cells is Flag-Streptavidin-HA (FSH)-tagged (arrowhead), leading to the mobility shift compared to parental HT1080 expressing endogenous GPX4 (arrow).

FIG. 12: HT1080 GPX4-knockout (KO) cells were grown in the presence of the radical trapping agent α-tocopherol (α-toco, 100 μM) to prevent cell death by unrestrained lipid peroxidation, or α-tocopherol was washed out at t=0, and cells were maintained in the absence (DMSO solvent control) or presence of CPBA (100 μM) over five consecutive days. Cell viability was assessed at the indicated time points using AquaBluer. Shown are mean values±S.E.M. of n=5 independent experiments. Treatment of cells with CPBA accelerates ferroptosis in the absence of GPX4. *, p<0.05.

FIG. 13: Effect of CPBA on ferroptotic cell death in GPX4-deficient HT1080 cells whose survival depends on the expression of FSP1. GPX4 knockout cells overexpressing FSP1 (GPX4-KO/FSP1-OE), or GPX4-KO cells overexpressing FSP1 and re-expressing GPX4 (GPX4-KO/FSP1-OE/GPX4-OE) were incubated with serial dilutions of CPBA. Note that no ferroptosis inducers (FINs) were added. GPX4-KO, FSP1-dependent cells die in a CPBA concentration-dependent manner, whereas cell death is prevented by liproxstatin-1 (lip-1) or GPX4 add-back.

FIG. 14: Western blot of cell line FSP1-KO.

FIG. 15: Effect of CPBA in comparison with the small molecule FSP1 inhibitor iFSP1 on FIN56-induced ferroptosis in FSP1-WT or FSP1-KO HT1080 cells. The extent of CPBA sensitization to ferroptosis is comparable to the effect of FSP1-deficiency. The insets on the right show FSP1-WT or FSP1-KO cells treated with either iFSP1 (top panel) or with CPBA (bottom panel). The curves of inhibitor-treated FSP1-WT cells are given for a reference, and are identical to the respective curves shown in the left panel. The ferroptosis-sensitizing effect of iFSP1 and CPBA is comparable to the effect of FSP1-loss, and no additional effect of iFSP1 or CPBA is observed in FSP1-deficient cells.

FIG. 16, FIG. 17: Effect of CPBA in comparison with the small molecule FSP1 inhibitor iFSP1 on BSO (FIG. 16) or RSL3 (FIG. 17)-induced ferroptosis in FSP1-WT or FSP1-KO HT1080 cells. The extent of CPBA sensitization to ferroptosis is comparable to the effect of FSP1-deficiency. The insets on the right show FSP1-WT or FSP1-KO cells treated with either iFSP1 (top panel) or with CPBA (bottom panel). The curves of inhibitor-treated FSP1-WT cells are given for a reference, and are identical to the respective curves shown in the left panel. The ferroptosis-sensitizing effect of iFSP1 and CPBA is comparable to the effect of FSP1-loss, and no additional effect of iFSP1 or CPBA is observed in FSP1-deficient cells. Data are means±S.E.M. of n≥3 independent experiments. Cell viability was assessed with Aquabluer after 16 h incubation with the indicated compounds. Final concentrations of iFSP1 or CPBA were 10 μM or 100 μM, respectively.

FIG. 18A: Effect of FSP1 membrane targeting on iFSP1 or CPBA-dependent ferroptosis sensitization. HT1080 FSP1-KO cells reconstituted with either FSP1-WT or the membrane binding-incompetent myristoylation mutant FSP1-G2A were incubated with serial FIN56-dilutions in the absence or presence of the DMSO solvent control, iFSP1 (10 μM), or CPBA (100 μM). iFSP1 or CPBA sensitize FSP1-proficient cells to ferroptosis only if FSP1 can be targeted to membranes.

FIG. 18B: Comparison of iFSP1 and CPBA effects on ferroptosis sensitivity in LOX-IMVI human melanoma cells. LOX-IMVI cells are FSP1-deficient, and were reconstituted with either mock vector (expressing Venus), or with FSP1-Venus, and treated as indicated.

FIG. 19: In vitro FSP1 activity assays. Shown are NADH consumption assays using recombinant, purified human or murine FSP1 (hFSP1 or mFSP1, respectively), and resazurine or coenzyme-Q1 (CoQ1) as electron acceptor molecules (top). The consumption of NADH was measured at 340 nm in a microtiter plate reader. Initial rates were determined, and data were fitted by linear regression. Shown are means±S.E.M. of n=3 independent experiments. Mean half-maximal inhibitory values (IC₅₀) are listed in the table (middle).

Determination of CPBA or iFSP1 binding kinetics using biolayer interferometry (bottom). N-terminally AviTag-fused murine or human FSP1 proteins were recombinantly expressed, purified, biotinylated and immobilized on streptavidin-coated biosensors. A Langmuir 1:1 model was used for steady state analyses, and a global exponential regression 1:1 model was used for kinetic analyses. Data are mean values±S.D. of at least four measurements.

FIG. 20: Concentration-dependent toxicity of FIN56 in a panel of (A) cancer cell lines or (B) immortalized, non-transformed hepatocytes (IHH, AML12) or a murine monocyte/macrophage cell line (RAW264.7). Cells were treated with DMSO solvent control, CPBA (100 μM) or iFSP1 (10 μM, except for 3 μM in HepG2 cells) in the absence or presence of liproxstatin-1 (lip-1, 100 nM). HCC, hepatocellular carcinoma. Cell viability was analyzed using AquaBluer after 16 h. Data are means±S.E.M. of n≥3 independent experiments.

FIG. 21: Concentration-dependent toxicity of FIN56 in (A) mouse embryonic fibroblasts (MEFs), or (B) Hepa 1-6 mouse hepatocellular carcinoma cells. Cells were treated with DMSO solvent control, or with the indicated concentrations of CPBA or iFSP1. Ferroptotic cell death was assessed by incubation in the absence or presence of liproxstatin-1 (lip-1; A, 500 nM; B, 250 nM). Cell viability was analyzed using Aquabluer after 16 h. Data are means±S.E.M. of n≥2 independent experiments.

FIG. 22: (A) Skeletal formulas of CPBA (here termed 1A) analogs, series 1 and 2. One of two 2-benzamidobenzoic acid moieties in CPBA is highlighted (circle).
(B) Effects of CPBA and its analogs on phosphoglycolate phosphatase (PGP) activity, using recombinant, purified murine PGP and phosphoglycolate (PG) as a substrate. Free inorganic phosphate released by PG dephosphorylation was quantified using malachite green. Compounds were initially tested at a concentration of 40 μM, and IC50 values were determined if the compound inhibited PGP activity by more than 50% compared to the DMSO solvent control. All results are mean values±S.D. of n=3 independent experiments; n.d., not determined.
(C, D) In vitro FSP1 activity assays with human (C) or murine (D) FSP1. Shown are NADH consumption assays using recombinant, purified human or mouse FSP1 (hFSP1, mFSP1) and resazurine or coenzyme-Q1 (CoQ1) as electron acceptor molecules. Compounds in the left panels (bar diagrams) were employed at a fixed concentration of 25 μM. In the right (FIG. 22C) or bottom (FIG. 22D) panels, the concentration-dependent inhibition of FSP1 was tested for selected compounds (cmps) as indicated.
(E) Effect of compound 2A (100 μM) on the concentration-dependent toxicity of FIN56 in two murine hepatocellular carcinoma cell lines. Cell viability was assessed using Aquabluer after 16 h. Ctrl, DMSO solvent control; lip-1, liproxstatin-1.

All data are means±S.E.M. of n≥3 independent experiments. Error bars not seen are hidden by the symbols.

### DETAILED DESCRIPTION OF THE INVENTION

The compounds of formula I can be prepared by standard processes of organic chemistry. For example, the compounds of formula I can be prepared by the processes described in EP 2 332 528 A1. Exemplarily, the compound of formula I may be a 2-(4-(4-(2-carboxyphenylcarbamoyl)phenoxy)benzamido) benzoic acid (CPBA) which can be prepared according to EP 2 332 528 A1 by converting 4,4′-oxydi (benzoylchlroide) with two equivalents of 2-aminobenzoic acid using Schotten-Baumann-conditions in the presence of organic or inorganic bases as acid scavengers. Furthermore, CPBA is commercially available.

Before the invention is described in more detail with respect to the preferred embodiments, the following general definitions are provided.

The present invention as illustratively described in the following may suitable be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”.

If hereinafter a group is defined to comprise at least a certain number of embodiments, this is also to be understood to disclose a group, which preferably consists only of these embodiments.

As used in this specification and in the appended claims, the singular form of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±10%, and preferably ±5%.

It is to be understood that the invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The term “compound(s) according to the invention”, or “compound(s) according to formula I” or “compound(s) of formula I” comprises the compound(s) as defined herein as well as a stereoisomer, salt, tautomer or N-oxide thereof. The term “compound(s) of the present invention” is to be understood to be equivalent to the term “compound(s) according to the invention”.

Depending on the substitution pattern, the compounds according to the invention may have one or more centers of chirality, in which case they are present as mixtures of enantiomers or diastereomers. The invention provides both the single pure enantiomers or pure diastereomers of the compounds according to the invention, and their mixtures. Suitable compounds according to the invention also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula I, i.e. to single enantiomers or diastereomers, as well as to mixtures thereof.

The compounds of formula I according to the present invention may be amorphous or may exist in one or more different crystalline states (polymorphs) which may have different macroscopic properties.

Salts of the compounds of formula I of the present invention are preferably “pharmaceutically acceptable” salts. The term “pharmaceutically acceptable salts” refers to a salt which possesses the effectiveness of the main compound and which is not biologically or otherwise undesirable, e.g. is neither toxic nor otherwise deleterious to the recipient thereof. Suitable salts of the compounds of formula I include but are not limited to acid addition salts, which may, for example, be formed by mixing a solution of the compound of formula I of the present invention with a solution of a pharmaceutically acceptable acid such as for example hydrochloric acid, sulfuric acid, acetic acid, trifluoroacetic acid, or benzoic acid. When the compounds of formula I of the present invention carry an acidic moiety, suitable pharmaceutically acceptable salts thereof can include alkali metal salts, alkaline earth metal salts and salts formed with suitable organic ligands such as quaternary ammonium salts.

The term “N-oxide” includes any compound of formula I, wherein a tertiary nitrogen atom is oxidized to an N-oxide moiety. “Tautomers” of the compounds of formula I may be present, if, e.g. any one of the substituents at the aromatic rings has tautomeric forms. Preferred tautomers include for example keto-enol tautomers.

The term “substituted” as used herein with regard to the compounds of formula I means that a hydrogen atom bonded to a designated atom is replaced with a specific substituent, provided that it results in a stable or chemically feasible compound. The term “substitutable” means that attached to the atom is a hydrogen, which can be replaced with a suitable substituent.

The organic moieties mentioned in the above definitions of the variables of the compounds according to formula I are collective terms for individual listings of the individual group members. The same applies to the term “halogen”. The term “Cn-Cm” indicates in each case the possible number of carbon atoms in the group.

The term “halogen” denotes in each case fluorine, bromine, chlorine and iodine.

The term “alkyl” as used herein with regard to the compound of formula I denotes in each case a straight chain or branched alkyl group having usually from 1 to 4 carbon atoms. Examples for preferred alkyl groups include methyl, ethyl, n-propyl, iso-propyl, n-butyl, 2-butyl, iso-butyl or tert-butyl.

The term “haloalkyl” as used herein with regard to the compound of formula I denotes in each case a straight chain or branched alkyl group having usually from 1 to 4 carbon atoms, wherein the hydrogen atoms of this group are partially or fully replaced with halogen atoms. Examples for haloalkyl moieties include but are not limited to fluoromethyl, difluoromethyl, trifluoromethyl, 1-fluoroethl, 2-fluoroethyl, or 2,2-difluoroethyl.

The term “alkenyl” as used herein with regard to the compound according to formula I denotes in each case an at least singly unsaturated hydrocarbon radical. In particular, it refers to an at least singly unsaturated hydrocarbon radical having at least one carbon-carbon double bond, having usually from 2 to 4 carbon atoms. Examples are vinyl, allyl, 1-propen-1-yl or 2-propen-2-yl.

The term “alkynyl” as used herein with regard to the compounds of formula I denotes in each case a hydrocarbon radical having at least one carbon-carbon triple bond and having usually from 2 to 4 carbon atoms. Examples are ethynyl, propargyl or 1-propyn-1-yl.

The term “cycloalkyl” as used herein with regard to the compounds of formula I denotes in each case a monocyclic cycloaliphatic radical having usually from 3 to 10 carbon atoms, preferably from 3 to 6 carbon atoms. Examples are cyclopropyl, cyclobutyl, cyclopentyl or cyclohexyl.

The term “carbocyclic” or “carbocyclyl” as used herein with regard to the compounds of formula I includes in general a 3- to 6-membered monocyclic ring comprising 3 to 6 carbon atoms. The carbocycle may be saturated, partially or fully unsaturated, or aromatic, wherein saturated means that only single bond is present and partially or fully unsaturated means that one or more double bonds are present in suitable positions, while the Hückel rule for aromaticity is not fulfilled. Aromatic means that the Hückel (4n+2) rule is fulfilled.

The term “heterocyclic” or “heterocyclyl” as used herein with regard to the compounds of formula I includes in general a 3- to 9-membered monocyclic ring, preferably a 3- to 6-membered monocyclic ring. The heterocycle may be saturated, partially or full unsaturated or aromatic, wherein saturated means that only single bond is present and partially or fully unsaturated means that one or more double bonds are present in suitable positions, while the Hückel rule for aromaticity is not fulfilled. Aromatic means that the Hückel (4n+2) rule is fulfilled. The heterocycle comprises one or more, e.g. 1, 2, 3 or 4 heteroatoms selected from N, O and S as ring members. The remaining ring members are carbon atoms.

The term “carbocyclylalkly” and “heterocyclylalkyl” as used herein refer to the corresponding carbocyclyl and heterocyclyl groups, which are bonded to the remainder of the molecule via an alkyl, preferably a C₁-C₄-alkyl group.

The term “treatment” as used herein includes also the option of “prophylaxis”. Thus, whenever reference is made herein to a “treatment” or “treating”, this is to be understood as “treatment and/or prophylaxis” or “treating and/or preventing”.

As has been set out above, the present invention relates in one aspect to a compound of formula I

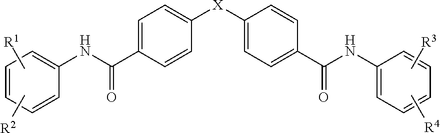

or a salt, tautomer, stereoisomer or N-oxide thereof,
wherein
X is O, S or CH₂;
R¹, R³ are independently of each other selected from H, halogen, CN, NO₂, C₁-C₄-alkyl, C₁-C₄-haloalkyl, C₂-C₆-alkenyl, C₂-C₆-alkynyl, OR^{a}, SR^{a}, C(═O)OR^{a}, NR^{b}R^{c}, and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
R², R⁴ are independently of each other selected from H, halogen, CN, NO₂, C₁-C₄-alkyl, C₁-C₄-haloalkylo, C₂-C₆-alkenyl, C₂-C₆-alkynyl, OR^{a}, SR^{a}, NR^{b}R^{c}; and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
and wherein
R^{a} is H, C₁-C₄-alkyl, C₁-C₄-haloalkyl, or C₂-C₆-alkenyl;
R^{b}, R^{c} are independently of each other selected from H, C₁-C₄-alkyl, and C₂-C₆-alkenyl;
R^{x} is halogen, CN, NO₂, C₁-C₄-alkyl, or C₁-C₄-haloalkyl,
for use in the treatment of cancer.

Preferred embodiments regarding the compounds of formula I, which are relevant for all aspects of the invention, are defined hereinafter.

In a preferred embodiment, the present invention relates to a compound of formula I for use in the treatment of cancer, the compound of formula I being

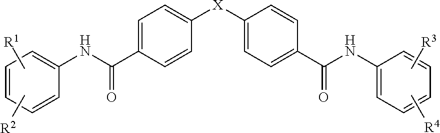

or a salt, tautomer, stereoisomer or N-oxide thereof,
wherein
X is O, S or CH₂.

In a more preferred embodiment of the present invention,
X is O or CH₂.

In a particularly preferred embodiment of the present invention,
X is O.

In connection with the above preferred embodiments it is to be understood that R¹, R², R³ and R⁴ are as defined above or further below.

In another preferred embodiment of the present invention, in said compound of formula I

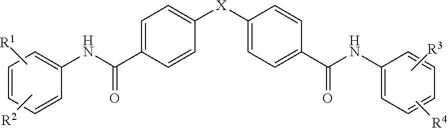

for use in the treatment of cancer,
R¹, R³ are independently of each other selected from H, halogen, CN, NO₂, C₁-C₄-alkyl, C₁-C₄-haloalkyl, C₂-C₆-alkenyl, C₂-C₆-alkynyl, OR^{a}, SR^{a}, C(═O)OR^{a}, NR^{b}R^{c}, and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
wherein
R^{x} is halogen, CN, NO₂, C₁-C₄-alkyl, or C₁-C₄-haloalkyl.

In connection with the above preferred embodiment it is to be understood that X, R², R⁴, R^{a}, R^{b} and R^{c} are as defined above or further below.

In a more preferred embodiment of the invention, in said compound of formula I
R¹, R³ are independently of each other selected from H, halogen, CN, NO₂, C₁-C₄-alkyl, and C(═O)OR^{a}.

In an even more preferred embodiment of the invention, in said compound of formula I, R¹, R³ are independently of each other selected from H, halogen, and C(═O)OR^{a}.

In a particularly preferred embodiment of the invention, in said compound of formula I R¹, R³ are C(═O)OR^{a}.

In connection with the above preferred embodiments it is to be understood that
R^{a} is H, C₁-C₄-alkyl, C₁-C₄-haloalkyl, or C₂-C₆-alkenyl,
preferably
R^{a} is H, or C₁-C₄-alkyl,
even more preferably
R^{a} is H.

In another preferred embodiment of the present invention, in said compound of formula I

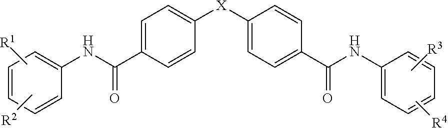

or a salt, tautomer, stereoisomer or N-oxide thereof,
for use in the treatment of cancer,
R², R⁴ are independently of each other selected from H, halogen, CN, NO₂, C₁-C₄-alkyl, C₁-C₄-haloalkyl, C₂-C₆-alkenyl, C₂-C₆-alkynyl, OR^{a}, SR^{a}, NR^{b}R^{c}; and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
wherein
R^{x} is halogen, CN, NO₂, C₁-C₄-alkyl, or C₁-C₄-haloalkyl.

In connection with the above preferred embodiment it is to be understood that X, R¹, R³, R^{a}, R^{b} and R^{c} are as defined above or further below.

In a more preferred embodiment of the invention, in said compound of formula I
R², R⁴ are independently of each other selected from H, halogen, CN, NO₂, and C₁-C₄-alkyl.

In an even more preferred embodiment of the present invention, in said compound of formula I
R², R⁴ are H.

Accordingly, in one embodiment, the present invention relates to a compound according to formula I

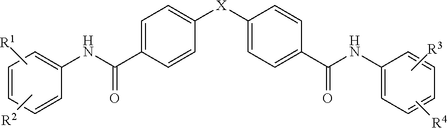

or a salt, tautomer, stereoisomer or N-oxide thereof,
for use in the treatment of cancer,
wherein X, R¹, R², R³, R⁴ and R^{a} have the following preferred meanings:
X is O;
R¹, R³ are independently of each other selected from H, halogen and C(═O)OR^{a};
R², R⁴ are independently of each other selected from H, halogen, CN, NO₂, and C₁-C₄-alkyl;
R^{a} is H, or C₁-C₄-alkyl.

In another embodiment, the present invention relates to a compound according to formula I

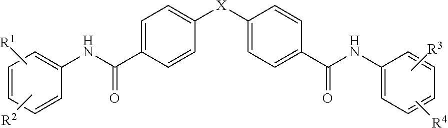

or a salt, tautomer, stereoisomer or N-oxide thereof,
for use in the treatment of cancer,
wherein X, R¹, R², R³, R⁴ and R^{a} have the following preferred meanings:
X is O;
R¹, R³ are C(═O)OR^{a};
R², R⁴ are H,
R^{a} is H, or C₁-C₄-alkyl.

In a preferred embodiment of the present invention, said compound of formula I

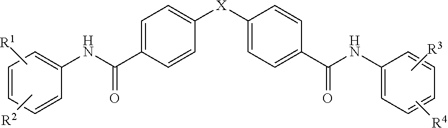

or a salt, tautomer, stereoisomer or N-oxide thereof,
for use in the treatment of cancer,
is preferably a compound according to the following formula

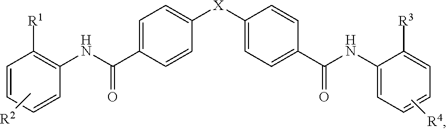

or a pharmaceutically acceptable salt thereof,
wherein
X is O;
R¹, R³ are C(═O)OR^{a};
R², R⁴ are H,
R^{a} is H, or C₁-C₄-alkyl.

Thus, the compound according to formula I is preferably a compound according to the following formula

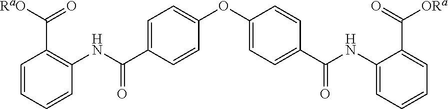

or a pharmaceutically acceptable salt thereof,
for use in the treatment of cancer,
wherein
R^{a} is H, or C₁-C₄-alkyl.

In a particularly preferred embodiment of the present invention, the compound of formula I is a compound according to the following formula

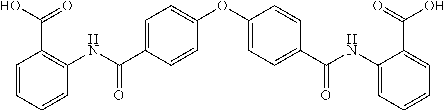 [SMILES: O=C(Nc1ccccc1C(=O)O)c1ccc(Oc2ccc(C(=O)Nc3ccccc3C(=O)O)cc2)cc1]

or a pharmaceutically acceptable salt thereof,
for use in the treatment of cancer.

In connection with the above depicted compound according to formula I, it is to be understood that said compound is also known with regard to its IUPAC name 2-[[4-[4-[(2-carboxyphenyl)carbamoyl]phenoxy]benzoyl]amino]-benzoic acid (CPBA, see FIG. 22A, compound 1A).

Accordingly, in a particularly preferred embodiment of the present invention, the compound of formula I is 2-[[4-[4-[(2-carboxyphenyl)carbamoyl]phenoxy]benzoyl]amino]-benzoic acid for use in the treatment of cancer.

The inventors of the present invention surprisingly found that the compounds according to formula I, in particular the compound according to the following formula

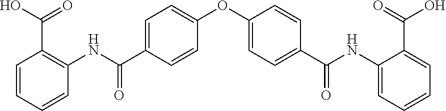 [SMILES: O=C(Nc1ccccc1C(=O)O)c1ccc(Oc2ccc(C(=O)Nc3ccccc3C(=O)O)cc2)cc1]

are capable of modulating FSP1, in particular to inhibit FSP1 and are therefore useful for the treatment of cancer.

The term “FSP1” relates to the Ferroptosis-Suppressor-Protein-1, also termed Apoptosis Inducing Factor Mitochondria Associated 2 (AIFM2) (NCBI Entrez Gene: 84883; Ensembl: ENSG00000042286; UniProtKB/Swiss-Prot: Q9BRQ8). Ferroptosis is a non-apoptotic form of cell death. Apoptosis is a programmed form of cell death, which is induced by the activation of signaling lines of proteins, the so-called caspases. In contrast thereto, ferroptosis relies on an oxidative process, which is dependent on cellular iron. This leads to the establishment of hydroxyl radicals, which react with unsaturated acyl chains of phospholipids in the cellular membrane. In a toxic amount, this leads to the progressive degradation of further membrane lipids and results in cellular death due to permeabilisation. Ferroptosis is phospholipid dependent, since the hallmark of ferroptosis is the iron-dependent accumulation of oxidatively damaged phospholipids (i.e., lipid peroxides). The term “ferroptosis-sensitive cancer” or “ferroptosis-sensitive cancer cell line” is used herein to describe a cancer or cancer cell line in which ferroptosis can be induced.

In one embodiment, the compound according to formula I as defined above or a pharmaceutically acceptable salt thereof is for use in the treatment of cancer, wherein treatment with said compound according to formula I as defined above or a pharmaceutically acceptable salt thereof sensitizes cells to ferroptosis inducer (FIN)-induced ferroptosis, optionally wherein the ferroptosis inducer is selected from the group consisting of erastin, buthionine sulfoximine (BSO), (1S/3R)-RSL3 (RSL3) or ferroptosis inducer 56 (FIN56). Erastin may be an erastin derivate, such as imidazole ketone erastin.

In one embodiment, the compound according to formula I as defined above or a pharmaceutically acceptable salt thereof is for use in the treatment of cancer, wherein treatment with said compound according to formula I as defined above or a pharmaceutically acceptable salt thereof sensitizes cells to FIN56-induced ferroptosis.

In one embodiment, the compound according to formula I as defined above or a pharmaceutically acceptable salt thereof is for use in the treatment of cancer, wherein treatment with said compound according to formula I as defined above or a pharmaceutically acceptable salt thereof sensitizes cells to erastin-induced ferroptosis. Erastin may be an erastin derivate, such as imidazole ketone erastin

In one embodiment, the compound according to formula I as defined above or a pharmaceutically acceptable salt thereof is for use in the treatment of cancer, wherein treatment with said compound according to formula I as defined above or a pharmaceutically acceptable salt thereof sensitizes cells to RSL3-induced ferroptosis.

In one embodiment, the compound according to formula I as defined above or a pharmaceutically acceptable salt thereof is for use in the treatment of cancer, wherein treatment with said compound according to formula I as defined above or a pharmaceutically acceptable salt thereof sensitizes cells to BSO-induced ferroptosis.

FSP1 suppresses ferroptosis by inhibiting lipid peroxidation. Accordingly, compounds which are capable of modulating FSP1, in particular compounds which inhibit FSP1 are useful in the treatment of cancer. The inventors of the present invention have surprisingly found that the compounds according to formula I can act as inhibitors of FSP1 and are therefore useful in the treatment of cancer.

In one embodiment, the compound according to formula I as defined above or a pharmaceutically acceptable salt thereof is for use in the treatment of cancer, wherein treatment with said compound according to formula I as defined above or a pharmaceutically acceptable salt thereof increases lipid peroxidation in a cell or cell sample.

In one embodiment, the compound according to formula I as defined above or a pharmaceutically acceptable salt thereof is for use in the treatment of cancer, wherein treatment with said compound according to formula I as defined above or a pharmaceutically acceptable salt thereof increases the amount of polyunsaturated fatty acid-triacylglycerides and/or reduces the amount of polyunsaturated fatty acid ether lipids in a cell or cell sample.

Cancer cells, in particular therapy-resistant cancer cells and those in the mesenchymal, metastasis-prone state are highly vulnerable to ferroptosis. The term “metastasis” describes the spread of cancer cells outside of the primary cancer site, either by growing directly into surrounding tissues, by traveling through the bloodstream or through the lymph system and subsequently infiltrating secondary tissues. The term “mesenchymal” describes a tumor environment in which mesenchymal stem cells at the site of the primary cancer increase the metastatic potential of the cancer cells. Mesenchymal tumors also include epithelial tumors that have gained mesenchymal characteristics after undergoing epithelial-mesenchymal transition. Mesenchymal characteristics include loss of cell-cell contacts and increased single-cell migration, which lead to increased metastatic potential.

The term “persister cells” or “persister cancer cells” or “drug tolerant persister (DTP) cells” refers to cancer cells that evaded cell death from chemotherapy or targeted therapy by entering a reversible slow proliferation state known as the drug tolerant persister (DTP) state. The persister cell pool constitutes a reservoir from which drug-resistant tumors may emerge. Persister cancer cells are typically in a high mesenchymal, therapy-resistant cell state. Persister cells may be characterized by a dependency on GPX4 and/or FSP1-mediated ferroptosis inhibition. Thus, persister cells may be particularly vulnerable to ferroptosis.

In one embodiment of the present invention, the compound according to formula I as defined above or a pharmaceutically acceptable salt thereof is for use in inducing phospholipid-dependent ferroptosis in an in vitro cell sample.

In one embodiment, the cell sample is a tumor cell line, an organ slice, an organoid, a spheroid or an assembloid. The skilled person is aware of methods of obtaining and cultivating such cell samples.

A “cancer cell line” as used herein refers to a cell line derived from a mammalian cancer which is cultured in vitro. A cancer cell line may be a primary cancer cell culture, i.e. cells cultured from a cancer tissue biopsy, or an immortalized cell line that keeps dividing and growing over time.

An “organ slice” as used herein refers to organ tissue from a mammal cultured in vitro, usually cut into “slices” using a microtome.

An “organoid” as used herein refers to a simplified version of an organ produced in vitro in a three-dimensional culture system, derived from one or more cells from a healthy or diseased tissue, from induced pluripotent stem cells, or embryonic stem cells. An organoid typically self-organizes and differentiates into a three-dimensional shape that resembles the organ it is derived from.

A “spheroid” as used herein refers to a three-dimensional cell culture model of aggregated cells.

An “assembloid” as used herein refers to an in vitro cell model system in which organoids resembling distinct tissues are assembled to model aspects of interactions between tissues or organ regions. To form assembloids, organoids can be supplemented with structural cells, immune cells or blood vessels. Like organoids, assembloids can be derived from induced human pluripotent stem cells, cells from healthy or diseased tissues, or embryonic stem cells.

In one embodiment of the present invention, the cancer cell lines are mammalian cell lines. In a preferred embodiment, the cancer cell lines are human cancer cell lines and/or rodent cancer cell lines. In another preferred embodiment of the present invention, the cancer cell lines are human cancer cell lines. In another preferred embodiment of the present invention, the cancer cell lines are rodent cancer cell lines. In particular, the rodent cancer cell lines are rat cell lines. In another embodiment, the rodent cancer cell lines are murine cell lines.

In this regard, it has surprisingly been found by the inventors of the present invention that the compounds according to formula I, and even more particular the compound according to the following formula

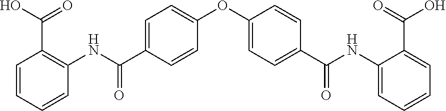 [SMILES: O=C(Nc1ccccc1C(=O)O)c1ccc(Oc2ccc(C(=O)Nc3ccccc3C(=O)O)cc2)cc1]

inhibit both human and rodent FSP1. Even more preferably, compounds according to formula I, preferably the compound according to the following formula

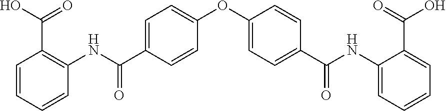 [SMILES: O=C(Nc1ccccc1C(=O)O)c1ccc(Oc2ccc(C(=O)Nc3ccccc3C(=O)O)cc2)cc1]

act as FSP1 inhibitor and are applicable in mammalian cancers, preferably human and/or rodent cancers.

Those compounds can advantageously be further refined and validated in genetically defined, immune-competent rodent tumor models for the evaluation of conventional, targeted, and immune therapies for cancer.

Thus, in one embodiment of the present invention, the compounds according to formula I as defined above are for use in the treatment of cancer.

The compounds according to formula I as defined above are for use in the treatment of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

With regard to the above embodiment, it is to be understood that the cancer can be of mammalian origin, preferably human or rodent origin. The compounds according to formula I as defined above are for use in the treatment of mammalian cancer, preferably human and/or rodent cancer.

In a more preferred embodiment of the present invention, the compounds according to formula I as defined above are for use in the treatment of human cancer.

In another more preferred embodiment of the present invention, the compounds according to formula I as defined above are for use in the treatment of rodent cancer.

In a further aspect, the present invention relates to the use of a compound or a pharmaceutically acceptable salt thereof as defined above for modulating FSP1 function in a cell. Modulating FSP1 function in a cell comprises contacting a cell capable of expressing FSP1 with an effective amount of the compound or a pharmaceutically acceptable salt thereof as defined above.

In a preferred embodiment of said use, the compound according to formula I as defined above acts as an antagonist of FSP1, thereby inducing ferroptosis in a cell capable of expressing FSP1. In other words, the compound according to formula I as defined above acts as an FSP1 inhibitor inducing ferroptosis.

In one embodiment, FSP1 is human FSP1 or rodent FSP1, preferably murine FSP1.

In another embodiment, the compound according to formula I as defined above acts as an antagonist of both human and murine FSP1.

In another aspect, the present invention relates to the use of a compound or a pharmaceutically acceptable salt thereof according to formula I as defined above in combination with one or more chemotherapeutic agents.

In another aspect, the present invention relates to a pharmaceutical composition comprising the compound according to formula I as defined above together with a pharmaceutically acceptable carrier and optionally further therapeutic excipient for use in the treatment of cancer, preferably wherein the further therapeutic excipient is a chemotherapeutic agent.

The pharmaceutical compositions can optionally comprise a suitable amount of a pharmaceutically acceptable carrier or a pharmaceutically acceptable excipient so as to provide the form for proper administration to a subject, cell line or ex vitro or in vitro cell sample. Such a pharmaceutical carrier or excipient can be a diluent, suspending agent, solubilizer, binder, disintegrant, preservative, coloring agent, lubricant, and the like.

The pharmaceutical carrier or excipient can be a liquid, such as water or an oil, including those of petroleum, animal, vegetable, or synthetic origin, such as peanut oil, soybean oil, mineral oil, sesame oil, and the like. The pharmaceutical carrier or excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable carrier or excipient is sterile when administered to an animal. Water is a particularly useful carrier or excipient when a compound of the present invention is administered intravenously. Saline solutions and aqueous dextrose and glycerol solutions can also be employed as liquid carriers or excipients, particularly for injectable solutions.

Suitable pharmaceutical carrier or excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The compositions, if desired, can also contain minor amounts of wetting or emulsifying agents, or pH buffering agents. The compositions can take the form of solutions, suspensions, emulsion, tablets, pills, pellets, multiparticulates, capsules, capsules containing liquids, powders, sustained release formulations, suppositories, emulsions, aerosols, sprays, suspensions, or any other form suitable for use.

In a preferred embodiment of the present invention, the pharmaceutical composition comprising the compound according to formula I as defined above together with a pharmaceutically acceptable carrier as defined above and optionally further therapeutic ingredients as defined above are for use in the treatment of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

In a preferred embodiment of the present invention, the pharmaceutical composition comprising the compound according to formula I as defined above together with a pharmaceutically acceptable carrier as defined above and optionally further chemotherapeutic agents are for use in the treatment of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

In one embodiment, the further chemotherapeutic agent is an antineoplastic agent, selected from alkylating agents, antimetabolites, topoisomerase inhibitors, antibiotics, mitotic inhibitors, and protein kinase inhibitors.

Examples of such chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomopholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY1 17018, onapristone, and toremifene (Fareston); aromatase inhibitors that inhibit the enzyme aromatase, which regulates estrogen production in the adrenal glands, such as, for example, 4 (5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Further suitable chemotherapeutic agents include anti-PDL1 monoclonal antibodies, such as atezolizumab, avelumab; anti-PD1 monoclonal antibodies, such as nivolumab, pembrolizumab; anti-CTLA4 monoclonal antibody, such as ipilimumab; anti-VEGFA monoclonal antibody, such as bevacizumab; anti-VEGFR2 monoclonal antibodies, such as ramucirumab, brivanib, orantinib; multikinase inhibitors, such as sorafenib, regorafenib, lenvatinib, cabozantinib, imatinib; EGFR inhibitors, such as neratinib, lapatinib, afatinib, trastuzumab, gefitinib, osimertinib; bifunctional fusion protein targeting TGF-β and PD-L1, such as bintrafusp alfa; anti-CD20 monoclonal antibody, such as Rituximab; proteasome inhibitor, such as Bortezomib; PEGylated L-Asparaginase, such as pegaspargase; CYP17A1 enzyme inhibitor, such as abiraterone; glucocorticoid, such as dexamethasone or prednisone. In a particularly preferred embodiment of the present invention, the pharmaceutical composition as defined above comprises a compound according to formula I, which is preferably a compound according to the following formula

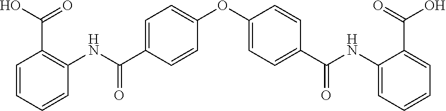 [SMILES: O=C(Nc1ccccc1C(=O)O)c1ccc(Oc2ccc(C(=O)Nc3ccccc3C(=O)O)cc2)cc1]

or a pharmaceutically acceptable salt thereof, for use in the treatment of cancer, preferably for use in the treatment of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

In another aspect, the present invention relates to the use of a compound according to formula I as defined above for the manufacture of a medicament for the treatment and/or prophylaxis of cancer.

In one embodiment, the present invention relates to the use as defined above for the manufacture of a medicament for the treatment and/or prophylaxis of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

### EXAMPLES

### Consumables and Reagents

All reagents were of the highest available purity. Reagents were purchased from the following providers: Sigma Aldrich (erastin, buthionine sulfoxamine/BSO, (1S/3R)-RSL3, deferoxamine/DFO, liproxstatin-1/lip-1, ferrostatin-1/fer-1, carbobenzoxy-valyl-alanyl-aspartyl-[O-methyl]-fluoromethylketone/zVAD-FMK, α-tocopherol, coenzyme-Q1/coQ1, staurosporine, nocodazole, tunicamycin, paclitaxel, etoposide, menadione, rotenone, carbonyl cyanide 4-(trifluoromethoxy)phenyl-hydrazone/FCCP, carboplatin, T863/iDGAT1, PF-06424439/iDGAT2, 4′,6-diamidino-2-phenylindole/DAPI); MedChemExpress (FIN56, IFSP1);

Thermo Fisher Scientific/Molecular Probes (BODIPY 581/591-C11, BODIPY 493/503); Cayman Chemical (resazurine). CPBA (compound 1A), compounds 1B-D and 2A-D were obtained from ChemDiv.

In the further description of the examples and the corresponding disclosed Figures, it is to be understood that compounds 1C, 1D and 2A-2D are comparative compounds.

### Antibodies

Antibodies were purchased from the following providers: Merck Millipore (α-actin mAb1501); Cell Signaling Technology (α-tubulin clone DM1A, mouse mAb #3873; α-GAPDH clone 14C10 Rabbit mAb #2118); Santa Cruz Biotechnology (α-GPX4 clone E-12, sc-166570; α-ACSL4 clone A5, sc-271800). The rabbit polyclonal α-PGP antibody (Seifried et al., 2014) and the α-FSP1 monoclonal antibody (Doll et al., 2019) have been described previously.

### Molecular Cloning

Human or murine FSP1 (hFSP1, UniProt Q9BRQ8; mFSP1, Q8BUE4) were cloned into the Ncol and EcoRI restriction sites of pETM11. The AviTag peptide (GLNDIFEAQKIEWHE) was inserted at the respective N-termini (i.e., C-terminal of the His₆-tag and the TEV cleavage site in pETM11), using inverse PCR mutagenesis as described previously (Fairhead et al., 2015). cDNAs were amplified using Q5 Hot Start High-Fidelity DNA Polymerase (NEB). All constructs were verified by sequencing.

### Expression and Purification of Recombinant FSP1

pETM11 constructs encoding N-terminally His₆-tagged hFSP1 or mFSP1 (with or without AviTag) were transformed into chaperone competent Escherichia coli (BL21 pG-Tf2, Takara Bio Inc.), and expressed for 20 h at 20° C. after induction with 0.2 mM isopropyl β-D-1-thiogalactopyranoside. Cells were harvested at 8,000×g for 15 min and resuspended in TNM (50 mM triethanolamine, 250 mM NaCl, 5 mM MgCl₂; PH 7.4) supplemented with protease inhibitors (complete EDTA free protease inhibitor cocktail; Roche). All purification steps were carried out at 4° C. Cells were lysed by sonication after addition of 1 mg/mL lysozyme and 50 μg/mL DNAse I, and cell debris was removed by centrifugation (14,000×g, 40 min, 4° C.). Cleared lysates were re-centrifuged (14,000×g, 20 min, 4° C.), supernatants were supplemented with 20 mM imidazole, and His₆-tagged hFSP1 or mFSP1 were purified by immobilized-metal affinity chromatography using a 5 mL HisTrap crude FF column (GE Healthcare) operated on an ÄKTA Purifier liquid chromatography system (GE Healthcare). Proteins were eluted with a linear 20-500 mM imidazole gradient in TNM. Peak fractions containing FSP1 were pooled, and the His₆-tag was cleaved with TEV protease (10 U/100 μg FSP1) at 4° C. during dialysis against TNM for 48 h (Slide-A-Lyzer G2 Dialysis Cassettes 10K MWCO; Thermo Fisher Scientific). Afterwards, His₆-tagged TEV protease and uncleaved FSP1 were removed with a HisTrap HP column. The flow through containing untagged FSP1 was concentrated (10 kDa MWCO; Amicon Ultra-15, Millipore). Samples were centrifuged at 30,000×g for 30 min at 4° C. to remove aggregates, and further purified on a Superdex 200 10/300 GL size exclusion chromatography column (GE Healthcare). Proteins were eluted in TNM, fractions containing pure FSP1 were pooled, concentrated and stored at −80° C. Protein concentrations were determined with the Micro BCA Protein Assay Kit (Thermo Fisher Scientific).

### Biotinylation of AviTag-FSP1

Purified, AviTag-fused mFSP1 or hFSP1 were enzymatically biotinylated with the E. coli biotin ligase BirA. To this end, FSP1 proteins (48 μM) were incubated with purified GST-BirA in the presence of ATP and D-biotin (Sigma Aldrich) as described (Fairhead et al., 2015). GST-BirA was removed using glutathione Sepharose 4 Fast Flow (GE Healthcare), and samples were dialyzed against TNM (pH 7.4) to remove free biotin.

### Determination of Ligand Binding Kinetics Using Biolayer Interferometry

Biotinylated, AviTag-fused mFSP1 or hFSP1 (1.5 μM in TNM buffer supplemented with 0.005% (v/v) Tween-20) was immobilized on streptavidin-coated biosensors (Super Streptavidin/SSA Dip and Read Biosensors, ForteBio). The sensors were then quenched with 27 μM biocytin (Sigma Aldrich), and binding kinetics were measured at 25° C. on a ForteBio Octet Red 96e instrument. Dissociation was initiated after 90 s. Reference sensors were quenched with 27 μM biocytin. Raw data was double referenced, and a global exponential regression 1:1 model was applied for kinetic analyses. Data are mean values±S.D. of at least n=4 measurements. K_{D}, dissociation constant; Kₒₙ, on-rate constant; K_{off}, off-rate constant.

### In Vitro FSP1 Oxidoreductase Assays

FSP1 activity assays were performed using recombinant, purified murine or human FSP1, NADH, and resazurine or coenzyme-Q1 (coQ1) as electron acceptor molecules. The following conditions were used: 200 μM resazurine were reduced with 750 nM mFSP1 or 150 nM hFSP1; 100 μM coQ1 was reduced with 400 nM mFSP1; and 137 μM coQ1 were reduced with 200 nM hFSP1. The consumption of NADH (500 μM) was measured at 340 nm in phosphate-buffered saline (80 mM Na₃PO₄, 300 mM NaCl; pH 7.0) at room temperature on a CLARIOstar microplate reader (BMG Labtech). Initial rates were determined, and data were fitted by linear regression using GraphPad Prism software, version 9.1.2.

### In Vitro Phosphoglycolate Phosphatase Assays

Recombinant, purified murine phosphoglycolate phosphatase (PGP) was expressed and purified as described (Seifried et al., 2014). Experiments were performed in TNM30 (30 mM triethanolamine, 30 mM NaCl, 5 mM MgCl₂; PH 7.5), supplemented with 0.01% (v/v) Triton X-100 and 5 mM dithiothreitol (DTT). Assays were conducted at room temperature in a final reaction volume of 50 μL in 96-well microtiter plates. PGP (5.7 nM) was pre-incubated with the compounds or the DMSO solvent control for 10 min, and enzyme reactions were started by the addition of the substrate phosphoglycolate (PG, 200 μM). Buffer without enzyme served as a background control. Inorganic phosphate release from PG was detected with malachite green solution (Biomol Green; Enzo Life Sciences). The absorbance at 620 nm (A620) was measured on an Envision 2104 multilabel microtiter plate reader (Perkin Elmer Life Sciences). Released phosphate was determined by converting the values to nmol Pi with a phosphate standard curve. Data were analyzed with GraphPad Prism version 7.0.5. For IC₅₀ determinations, loginhibitor versus response was calculated for a Hill slope of −1.

### Cell Culture, CRISPR/Cas9 and RNA Interference

Cell culture media and supplements were from Thermo Fisher Scientific, unless specified otherwise. Mouse embryonic fibroblasts (MEFs) were generated from Pgp-wildtype (Pgp^{fl/fl}) or heterozygous Pgp-inactivated mouse embryos (Pgp^{fl/D34N}) as described (Segerer et al., 2016). Human embryonic kidney HEK-AD293 (HEK293) cells (Stratagene), HT-1080 human fibrosarcoma cells, PC3 and Du145 human prostate carcinoma cells, HepG2 human hepatocytic carcinoma (HCC) cells, Hepa 1-6 murine HCC, 4T1 mouse breast carcinoma, Colon-26 mouse colon carcinoma, C6 rat glioblastoma and RAW264.7 mouse monocytes/macrophages (all from LGC Standards) were cultured in Dulbecco's modified Eagle's medium (DMEM, 4.5 g/L glucose) supplemented with 10% (v/v) heat-inactivated fetal calf serum (FCS), 2 mM L-glutamine, 100 U/mL penicillin and 100 μg/mL streptomycin (Pen/Strep). Hepa-1c1c7 murine HCC were grown in alpha MEM without nucleosides (Gibco, cat. no. 31095029) supplemented with 10% FCS and Pen/Strep. JTC-27 rat HCC were cultured in RPMI-1640 with 10% FCS and Pen/Strep. IHH immortalized human hepatocytes were cultured on collagen-coated (50 μg/mL rat tail collagen-I; Sigma Aldrich) dishes in Williams E Medium (Sigma Aldrich, cat. no. W1878) supplemented with 10% FCS, 2 mM L-glutamine, Pen/Strep, 20 U/L insulin (Sigma Aldrich) and 50 nM dexamethasone (Sigma Aldrich). AML-12 (a hepatocyte cell line derived from a mouse transgenic for TGFα) were cultured in DMEM/F12 medium with GlutMAX (Gibco, cat. no. 31331028) supplemented with 10% FCS, Pen/Strep, insulin-transferrin-selenium (1:100 dilution of ITS-G supplement stock solution; Gibco, cat. no. 41400045) and 40 ng/mL dexamethasone.

The following HT1080 fibrosarcoma cell lines were generated using CRISPR/Cas9 technology, as previously described (Doll et al., 2019): HT1080 GPX4-knockout (KO) cells (maintained in the presence of 100 μM α-tocopherol); HT1080 GPX4 knockout cells overexpressing FSP1 (GPX4-KO/FSP1-OE); GPX4-KO cells overexpressing FSP1 and re-expressing GPX4 (GPX4-KO/FSP1-OE/GPX4-OE); HT1080 FSP1-WT or FSP1-KO cells; and HT1080 FSP1-KO cells reconstituted with either FSP1-WT or FSP1-G2A. LOX-IMVI (BRAF V600E heterozygous human melanoma cells) expressing either the Venus mock vector or FSP1-Venus (Doll et al., 2019) were grown in RPMI-1640 supplemented with 10% FCS, 2 mM L-glutamine and Pen/Strep.

### Lipid Droplet Staining

HT1080 cells were seeded on glass coverslips (15,000 cells per well of 12-well plate) in complete DMEM, and incubated for 16 h with 100 μM CPBA or with 0.1% of the DMSO solvent control, in the absence or presence of inhibitors of DGAT1 and DGAT2 (10 μM T863/iDGAT1; 5 μM PF-06424439/iDGAT2). Cells were fixed with 4% para-formaldehyde, lipid droplets were stained with the neutral lipid droplet stain BODIPY 493/503 (2 μM, 30 min), and nuclei were counterstained with 4′,6-diamidino-2-phenylindole (DAPI). Cells were mounted with Immu-Mount (Fisher Scientific), and imaged by fluorescence microscopy on a THUNDER imager (THUNDER Imager Live Cell & 3D Cell Culture & 3D Assay system, Leica DMI8; Leica Microsystems), using a 63× objective.

### Western Blotting

Cultured cells were lysed in 50 mM Tris, pH 7.5; 150 mM NaCl; 1% (v/v) Triton X-100; 0.5% (w/v) sodium deoxycholate; 0.1% (w/v) sodium dodecyl sulfate; 5 μg/mL aprotinin, 1 μg/mL leupeptin, 1 μg/mL pepstatin, and 1 mM 4-(2-aminoethyl)benzenesulfonyl fluoride (Pefabloc). Protein concentrations were determined using the Micro BCA Protein Assay Kit (Thermo Fisher Scientific). Proteins were separated by SDS-PAGE, transferred to nitrocellulose membranes by semidry-blotting, and incubated with the indicated antibodies.

### Cell Viability Assays

Cells were seeded onto 96-well plates (5,000 cells per well), and treated with serial dilutions of ferroptosis-inducing compounds (erastin, BSO, RSL3, FIN56) or cytotoxic drugs (staurosporine, nocodazole, tunicamycin, paclitaxel, etoposide, menadione, rotenone, FCCP, carboplatin) in the absence or presence of CPBA or iFSP1 (100 μM or 10 μM, respectively; unless stated otherwise) or the DMSO solvent control. Ferroptotic cell death was evaluated by incubating in the absence or presence of liproxstatin-1 (100 nM), ferrostatin-1 (200 nM), or deferoxamine (100 μM). Cell viability was assessed after 16 h using AquaBluer as an indicator of viable cells according to the manufacturer's recommendations (MultiTarget Pharmaceuticals, LLC). Fluorescence (excitation, 540 nm; emission, 590 nm) was analyzed on a Clariostar microplater reader.

Assessment of Lipid Peroxidation with Flow Cytometry

Approximately 10,000 HT1080 cells were seeded per well of a 48-well dish. The next day, cells were treated for 16 h with DMSO (0.1%, solvent control), CPBA (100 μM), or liproxstatin-1 (100 nM) in the absence or presence of subeffective concentrations of RSL3 (50 nM), FIN56 (50 nM), or BSO (1 μM). Cells were incubated with the BODIPY 581/591-C11 probe (1 μM) for 30 min at 37° C. in a tissue culture incubator, harvested by trypsinization, and resuspended in 500 μL phosphate-buffered saline supplemented with 0.5 mg/mL soybean trypsin inhibitor (Sigma Aldrich). Cells were analyzed using the 488-nm laser of a flow cytometer (FACSCalibur, BD Biosciences) for excitation. Data were collected from the FL1 detector with a 530 nm band-pass filter. At least 5,000 cells were analyzed per sample. Data analysis was conducted using FlowJo Software.

### Lipidomics

### Lipid Extraction for Mass Spectrometry Lipidomics

Mass spectrometry-based lipid analysis was performed by Lipotype GmbH (Dresden, Germany) as described (Sampaio et al. 2011). Lipids were extracted using a two-step chloroform/methanol procedure (Ejsing et al. 2009). Samples were spiked with internal lipid standard mixture containing: cardiolipin 16:1/15:0/15:0/15:0 (CL), ceramide 18:1;2/17:0 (Cer), diacylglycerol 17:0/17:0 (DAG), hexosylceramide 18:1;2/12:0 (HexCer), lyso-phosphatidate 17:0 (LPA), lyso-phosphatidylcholine 12:0 (LPC), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG), phosphatidylinositol 16:0/16:0 (PI), phosphatidylserine 17:0/17:0 (PS), cholesterol ester 20:0 (CE), sphingomyelin 18:1;2/12:0;0 (SM), triacylglycerol 17:0/17:0/17:0 (TAG). After extraction, the organic phase was transferred to an infusion plate and dried in a speed vacuum concentrator. First step dry extract was re-suspended in 7.5 mM ammonium acetate in chloroform/methanol/propanol (1:2:4, V:V:V) and second step dry extract in 33% ethanol solution of methylamine in chloroform/methanol (0.003:5:1;V:V:V). All liquid handling steps were performed using Hamilton Robotics STARlet robotic platform with the Anti Droplet Control feature for organic solvents pipetting.

### MS Data Acquisition

Samples were analyzed by direct infusion on a QExactive mass spectrometer (Thermo Fisher Scientific) equipped with a TriVersa NanoMate ion source (Advion Biosciences). Samples were analyzed in both positive and negative ion modes with a resolution of Rm/z=200=280000 for MS and Rm/z=200=17500 for MSMS experiments, in a single acquisition. MS/MS was triggered by an inclusion list encompassing corresponding MS mass ranges scanned in 1 Da increments (Surma et al. 2015). Both MS and MS/MS data were combined to monitor CE, DAG and TAG ions as ammonium adducts; PC, PC O—, as acetate adducts; and CL, PA, PE, PE O—, PG, PI and PS as deprotonated anions. MS only was used to monitor LPA, LPE, LPE O—, LPI and LPS as deprotonated anions; Cer, HexCer, SM, LPC and LPC O— as acetate adducts.

### Data Analysis and Post-Processing

Data were analyzed with in-house developed lipid identification software based on LipidXplorer (Herzog et al. 2011; Herzog et al. 2012). Data post-processing and normalization were performed using an in-house developed data management system. Only lipid identifications with a signal-to-noise ratio >5, and a signal intensity 5-fold higher than in corresponding blank samples were considered for further data analysis.

### Statistical Analysis

Western blots were quantified using the public domain NIH Image program (developed at the U.S. National Institutes of Health, http://rsb.info.nih.gov/nih-image/). Statistical analysis was performed using GraphPad Prism version 7.05 or 9.1.2 (GraphPad, San Diego, CA, USA).

The number of n independent experiments and the applied statistical tests are stated in the respective legends.

### Example 1: Identification of CPBA as a Ferroptosis Sensitizer

HT1080 cells, mouse embryonic fibroblasts (MEFs, non-transformed, non-immortalized) and human prostate carcinoma cell lines Du145 and PC3 were incubated with serial dilutions of ferroptosis inducers (FINs) erastin, BSO, RSL3 or FIN56 in the absence or presence of CPBA and/or the ferroptosis inhibitor liproxstatin-1 (HT1080, Du145, PC3: 200 μM CPBA, MEF: 100 μM CPBA; lip-1:100 nM), and cell viability was analyzed after 16 h using Aquabluer. Concentration-response curves were constructed by non-linear regression, and area-under-the-curve (AUC) values were calculated (AUC FIN+ctrl/AUC FIN+ctrl+lip-1 and AUC FIN+CPBA/AUC FIN+CPBA+lip-1). Cell viability assays were performed as described above. Exemplary concentration-response curves from HT1080 cells are shown in FIG. 1B. CPBA increases the negative effect of FINs on cell viability.

In order to quantify the effects of CPBA on induction of ferroptosis by FINs, area-under-the-curve (AUC) metrics of cell line sensitivity to CPBA in the presence of different FINs was analysed. Scoring was based on AUC values, because they reflect changes in IC₅₀ (potency) as well as in efficacy (% inhibition). The heatplot depicts the concentration-dependent synthetic lethality of CPBA when combined with FINs (see FIG. 1C). The lower the value, the stronger the CPBA-induced effect on FIN-induced ferroptosis. CPBA strongly increases FIN-induced ferroptosis in all cell lines.

Next, in order to test if ferroptosis-sensitization by CPBA is concentration-dependent, HT1080 and PC3 cells were incubated with serial dilutions of FINs±CPBA as described above, and cell viability was analyzed after 16 h. AUC values (AUC FIN+CPBA/AUC FIN+ctrl) were calculated as described above. The results show that the ferroptosis-sensitization by CPBA is concentration-dependent (see FIG. 1D).

To study the effect of increasing CPBA concentrations in the absence of ferroptosis inducers on the viability of HT1080, Du145 or PC3 cells. Cells were incubated with the indicated concentrations of CPBA alone or in the presence of ferroptosis inhibitors deferoxamine (DFO, an iron-chelator and ferroptosis inhibitor); ferrostatin (fer, a ferroptosis inhibitor); liproxstatin-1 (lip-1, a ferroptosis inhibitor); or zVAD-FMK (a pan-caspase and apoptosis inhibitor), and cell viability was analyzed after 16 h. The results show that CPBA alone is not cytotoxic at concentrations of up to 200 μM. Non-ferroptotic, non-apoptotic cell death is observed in the presence of 400 μM CPBA (see FIG. 1E).

### Example 2: Ferroptosis-Sensitization by CPBA is Independent of Phosphoglycolate Phosphatase (PGP)

Mammalian PGP is a haloacid dehalogenase-type phosphatase important for lipid metabolism and controls fatty acid flux through the intracellular triacylglycerol/fatty acid cycle (Segerer et al. 2018). In order to study the potential involvement of PGP in the sensitization of cells to ferroptosis induction, HT1080 cells were transiently transfected with small interfering RNA (SiRNA) oligonucleotides targeting Pgp mRNA or with control siRNA, using Lipofectamine 2000 (Seifried et al., 2014). For PGP overexpression, HT1080 were transfected with PGP^{WT} in pcDNA3, or with the empty vector control. Western Blot was performed as described above to verify overexpression or downregulation of PGP protein, respectively (see FIG. 2.). Cells were incubated with serial dilutions of FIN56, BSO or RSL3 and treated with DMSO solvent control or CPBA and cell viability was analyzed as described above (See FIGS. 2 and 3). Concentration-response curves were plotted as described above. CPBA was able to sensitize both cells transiently overexpressing PGP and cells with transiently downregulated PGP levels to FIN-induced ferroptosis.

The above experiment was repeated in mouse embryonic fibroblasts (MEFs) generated from Pgp-wildtype (Pgp^{fl/fl}) or heterozygous Pgp-inactivated mouse embryos (Pgp^{fl/D34N}). CPBA was able to sensitize both Pgp^{fl/fl} and Pgp^{fl/D34N} cells to FIN-induced ferroptosis (see FIG. 4).

The results show that CPBA sensitization of cells to FIN-induced ferroptosis was not changed during overexpression or depletion of PGP. Thus, it was surprisingly shown that ferroptosis-sensitization by CPBA is independent of PGP.

### Example 3: CPBA does not Generally Sensitize Cells to Death

In order to verify that CPBA sensitization was specific to ferroptosis-induced cell death, HT1080 fibrosarcoma (see FIG. 5) or PC3 prostate carcinoma cells (see FIG. 6) were incubated for 16 h with a panel of cytotoxic drugs (STS, staurosporine; NOC, nocodazole; TUN, tunicamycin; PAX, paclitaxel; ETO, etoposide; MEN, menadione; ROT, rotenone; FCCP, carbonyl cyanide 4-(trifluoromethoxy)phenyl-hydrazone; CPT, carboplatin; 11 serial 1:3 dilutions each), in the absence (DMSO solvent control) or presence of CPBA (100 μM), and cell viability was analyzed.

The results show that CPBA did not sensitize cells to non-ferroptotic cell death induced by cytotoxic drugs.

### Example 4: CPBA does not Affect Steady-State Expression Levels of Known Ferroptosis Regulators

Next, we tested whether CPBA sensitization affect the expression levels of the known ferroptosis regulators GPX4 (glutathione peroxidase-4), ACSL4 (acyl-CoA synthetase long chain family member 4) or FSP1 (ferroptosis suppressor protein-1).

HT1080 cells were cultured in the presence of liproxstatin-1 (100 nM), and incubated for 16 h with the solvent control DMSO (0.1%), RSL3 (1 μM), FIN56 (10 μM), or BSO (100 μM) in the presence of either 0.1% DMSO (−) or 100 μM CPBA (+). Cells were lysed and probed with the indicated antibodies as described above. Comparable protein loading was assessed by reprobing the same blots with actin- or tubulin-directed antibodies. Quantification of relative intensity of Western Blot bands was performed as described above (see FIG. 7).

The results show that the expression of GPX4, ACSL4 and FSP1 was not affected by the presence of CPBA (see FIG. 7).

### Example 5: CPBA Lowers Cellular Defense Against Lipid Peroxidation

Effects of CPBA on cellular lipid peroxidation were investigated using a BODIPY 581/591-C11 probe as described above. BODIPY 581/591-C11 is a fluorescent ratio probe of lipid oxidation and is used to detect reactive oxygen species. Oxidation of the polyunsaturated butadienyl portion of the dye results in a shift of the fluorescence emission peak from about 595 nm (nonoxidized form) to about 520 nm (oxidized form) (see FIG. 8).

HT1080 cells were incubated for 16 h with DMSO (0.1%, solvent control), CPBA (100 μM), or liproxstatin-1 (lip-1, 100 nM) in the absence or presence of subeffective concentrations of RSL3 (50 nM), FIN56 (50 nM), or BSO (1 μM). Fluorescence intensity was quantified by flow cytometry using a 530 nm band-pass filter as described above and quantified. Increased mean fluorescence intensity represents an increased in oxidized BODIPY 581/591-C11, which indicates an increase in lipid peroxidation.

The results show that in the presence CPBA, the amount of lipid peroxidation in the cells is increased. This effect of CPBA was not observed in the presence of liproxstatin-1.

Next, we analyzed the lipidomic signature of HT1080 cells treated with CPBA (200 μM, 16 h) using the lipidomics methods described above. We found that CPBA-treated cells downregulate polyunsaturated fatty acid (PUFA) ether lipids (PE-O, phosphatidylethanolamine ether; PC—O, phosphatidylcholine ether) and upregulate PUFA-triacylglycerides (PUFA-TAGS) (see FIG. 9). This lipidomic signature has recently been described in cancer cells that are initially dependent on GPX4 to evade ferroptosis, and indicates a sensitization to ferroptosis (Zou Y et al. Nature. 2020 September; 585 (7826): 603-608. doi: 10.1038/s41586-020-2732-8).

Lipid droplets serve as cellular stores for triglycerides and other neutral lipids such as cholesterol esters. Because genetic Pgp-inactivation or PGP knockdown can lead to the build-up of intracellular triglycerides in mice, rats, and murine cell lines (Mugabo, Y. et al. Identification of a mammalian glycerol-3-phosphate phosphatase: Role in metabolism and signaling in pancreatic β-cells and hepatocytes. Proc Natl Acad Sci USA 113, E430-9 (2016); Segerer, G. et al. An essential developmental function for murine phosphoglycolate phosphatase in safeguarding cell proliferation. Sci Rep 6, 35160 (2016); Segerer, G. et al. A phosphoglycolate phosphatase/AUM-dependent link between triacylglycerol turnover and epidermal growth factor signaling. Biochim Biophys Acta Mol Cell Biol Lipids 1863, 584-594 (2018).

We next investigated the impact of CPBA on triglyceride levels and ferroptosis sensitivity by first imaging lipid droplets with the neutral lipid stain BODIPY 493/503. As shown in FIG. 10, CPBA-treatment indeed triggered the accumulation of lipid droplets in HT1080 cells (compare DMSO solvent control in upper left panel with CPBA-treated cells in lower left panel: the small bright spots in the CPBA-treated cells represent lipid droplets).

The diglyceride acyltransferase isoforms 1 and 2 (DGAT1, DGAT2) are required to catalyze the formation of triglycerides from diacylglycerol and acyl-CoA. Blocking lipid droplet formation with inhibitors of DGAT1 (T863, IDGAT1; 10 μM) and DGAT2 (PF-06424439, IDGAT2; 5 μM) indeed blocked lipid droplet formation both in DMSO- and in CPBA-treated cells (see FIG. 10, upper right and lower right panels). However, the absence or presence of lipid droplets did not influence the CPBA-mediated sensitization of HT1080 cells to FIN56-induced cell death (see FIG. 10, right panel; compare CPBA with CPBA+iDGAT1/2). Hence, lipid droplets are dispensable for CPBA-induced sensitization to ferroptosis.

### Example 6: CPBA-Induced Sensitization to Ferroptosis is Dependent on FSP1

In order to study the effect of CPBA-induced sensitization to ferroptosis in the absence of the ferroptosis suppressor GPX4, we generated HT1080 GPX4-knockout (KO) cells using CRISPR/Cas9 technology as described above. Lack of GPX4 expression in GPX4-KO cell lines was confirmed by Western Blot as described above (see FIG. 11). These cells were grown in the presence of the radical trapping agent α-tocopherol (α-toco, 100 μM) to prevent cell death by unrestrained lipid peroxidation. α-Tocopherol was washed out at t=0, and cells were maintained in the absence (DMSO solvent control) or presence of CPBA (100 μM) over five consecutive days, control cells were maintained on α-tocopherol (See FIG. 12). Cell viability was assessed at 48 h, 72 h, 96 h and 120 h using AquaBluer.

The results show that treatment of cells with CPBA accelerates ferroptosis in the absence of GPX4.

Next, we tested the effect of CPBA on ferroptotic cell death in GPX4-deficient HT1080 cells whose survival depends on the expression of FSP1 in order to suppress ferroptosis in the absence of GPX4. GPX4 knockout cells overexpressing FSP1 (GPX4-KO/FSP1-OE), or GPX4-KO cells overexpressing FSP1 and re-expressing GPX4 (GPX4-KO/FSP1-OE/GPX4-OE) were incubated with serial dilutions of CPBA. Note that no ferroptosis inducers (FINs) were added. Protein expression was confirmed by Western Blot as described above (see FIG. 11).

GPX4-KO, FSP1-dependent cells die in a CPBA concentration-dependent manner, whereas cell death is prevented by liproxstatin-1 (lip-1) or GPX4 add-back (see FIG. 13). This result shows that surprisingly, FSP1 is sensitive to CPBA.

### Example 7: Comparison of CPBA and iFSP1

We next performed comparative experiments to compare the effect of CPBA with the effect of the small molecule FSP1 inihibitor iFSP1. To this end, we incubated FSP1-WT or FSP1-KO HT1080 cells with FIN56 (see FIG. 15), BSO (see FIG. 16) or RSL3 (see FIG. 17) to induce ferroptosis as described above. Lack of FSP1 expression in FSP1-KO cell lines was confirmed by Western Blot as described above (see FIG. 14). Comparing FSP1-WT+DMSO cells with FSP1-KO+DMSO cells, FSP1-WT+iFSP1 cells and FSP1-WT+CBPA cells, we found the extent of CPBA sensitization to ferroptosis is comparable to the effect of FSP1-deficiency, while the effect of iFSP1 was slightly smaller than the effect of CBPA in all conditions (see FIG. 15).

Next, we compared the treatment of FSP1-WT and FSP1-KO cells with either iFSP1 or with CPBA (see FIGS. 15, 16 and 17). The ferroptosis-sensitizing effect of iFSP1 and CPBA is comparable to the effect of FSP1-loss, and no additional effect of iFSP1 or CPBA is observed in FSP1-deficient cells.

These results show that the effect of treatment with CPBA closely resembles the effect of iFSP1 or loss of FSP1. There is no additive effect of CPBA and loss of FSP1, indicating that they are part of the same pathway.

### Example 8: Effect of FSP1 Membrane Targeting on iFSP1 or CPBA-Dependent Ferroptosis Sensitization

In order to study the effect of FSP1 membrane targeting on iFSP1 or CPBA-dependent ferroptosis sensitization, HT1080 FSP1-KO cells reconstituted with either FSP1-WT or the membrane binding-incompetent myristoylation mutant FSP1-G2A were incubated with serial FIN56-dilutions in the absence or presence of the DMSO solvent control, iFSP1 (10 μM), or CPBA (100 μM) (see FIG. 18A).

The results show that iFSP1 or CPBA sensitize FSP1-proficient cells to ferroptosis only if FSP1 can be targeted to membranes.

### Example 9: Comparison of iFSP1 and CPBA Effects on Ferroptosis Sensitivity in LOX-IMVI Human Melanoma Cells

LOX-IMVI cells are FSP1-deficient, and were reconstituted with either mock vector (expressing Venus), or with FSP1-Venus to restore FSP1 expression, and treated with either CPBA or iFSP1 as described above (see FIG. 18B).

The results show that CPBA does not enhance the ferroptosis-inducing effect of FIN56 in FSP1-deficient cells, indicating that FSP1 and the CPBA target are part of the same pathway. Furthermore, FSP1-expression rescues CPBA- and FIN56-treated cells from ferroptosis. As described previously (Doll et al., Nature 2019), iFSP1 is ineffective in LOX-IMVI cells.

### Example 10: CPBA is a Direct Inhibitor of Human and Murine FSP1

In order to determine inhibition of FSP1 by CPBA, FSP1 activity was measured in cells incubated with either CPBA or iFSP1 as a positive control. In vitro FSP1 oxidoreductase activity assays were performed using recombinant, purified murine or human FSP1, NADH, and resazurine or coenzyme-Q1 (coQ1) as electron acceptor molecules as described above (see FIG. 19).

The results show that CPBA inhibits human and murine FSP1 activity. Surprisingly, CPBA was also able to inhibit murine FSP1 activity, whereas iFSP1 has no effect on mFSP1.

We also determined the CPBA and iFSP1 on-rate, off-rate and dissociation constants, which are associated with the binding and unbinding reaction of target and inhibitor. N-terminally AviTag-fused murine or human FSP1 proteins were recombinantly expressed, purified, biotinylated and immobilized on streptavidin-coated biosensors. A Langmuir 1:1 model was used for steady state analyses, and a global exponential regression 1:1 model was used for kinetic analyses. The binding kinetics of CPBA and iFSP1 show similar affinities of both compounds for murine FSP1, whereas the affinity of iFSP1 for human FSP1 exceeds the affinity of CPBA for human FSP1. Thus, factors other than the parameters detectable by BLI analysis must contribute to the much more effective inhibition of murine FSP1 by CPBA compared to iFSP1.

### Example 11: CPBA Sensitizes Tumor Cell Lines from Human, Murine and Rat Origin to Ferroptosis

We measured the effect of CPBA-sensitization of tumor cell lines to FIN56-induced ferroptosis by incubation various cancer cell lines (FIG. 20A), or immortalized, non-transformed hepatocytes (IHH, AML12) or a murine monocyte/macrophage cell line (RAW264.7) (FIG. 20 B) with FIN56 as described above and treating cells with DMSO solvent control, CPBA (100 μM) or iFSP1 (10 μM, except for 3 μM in HepG2 cells) in the absence or presence of liproxstatin-1 (lip-1, 100 nM) (see FIG. 20). Cell viability was analyzed using AquaBluer after 16 h as described above.

Results show that CPBA, but not iFSP1-sensitizes cancer cell lines of different species and tumor types to ferroptosis. In contrast, the toxicity of FIN56 in non-transformed hepatocytic or in monocyte/macrophage-like cell lines was not markedly increased by the addition of CPBA or iFSP1. Hence, CPBA specifically sensitizes tumor cells to FIN-induced ferroptosis.

### Example 12: Non-Specific Toxicity of iFSP1 in Mouse Cell Lines

In order to test concentration-dependent toxicity of treatment with FIN56 and either CPBA or iFSP1, we treated mouse embryonic fibroblasts (MEFs, see FIG. 21A), or Hepa 1-6 mouse hepatocellular carcinoma cells (see FIG. 21B) with DMSO solvent control, or with the indicated concentrations of CPBA or iFSP1. Ferroptotic cell death was assessed by incubation in the absence or presence of liproxstatin-1 (lip-1; A, 500 nM; B, 250 nM).

Results show that concentrations >10 μM of iFSP1, but not of CPBA, are non-specifically toxic to cells. Furthermore, CPBA surprisingly sensitizes MEFs or Hepa 1-6 cells to FIN56-induced ferroptosis, while iFSP1 does not have an effect on mouse cell lines.

### Example 13: Structure-Activity Relationship Study with CPBA Analogs

We created two series (series 1 and series 2) of analogs of CPBA (see FIG. 22). In order to study the activity of different analogs, we tested the effects of CPBA and its analogs on PGP activity and on human and murine FSP1 as described above.

The skeletal formulas of CPBA (here termed 1A) analogs, series 1 and 2, are depicted in FIG. 22 A. We tested the ability of the CPBA analogs to inhibit FSP1 and used them as comparative examples to CPBA. We found that overall, none of the comparative CPBA analogs showed superior inhibition of FSP1 than CPBA itself.

Since CPBA is an inhibitor of PGP, we tested the effects of its analogs on phosphoglycolate phosphatase (PGP) activity, using recombinant, purified murine PGP and phosphoglycolate (PG) as a substrate. Free inorganic phosphate released by PG dephosphorylation was quantified using malachite green. Compounds were initially tested at a concentration of 40 μM, and IC₅₀ values were determined if the compound inhibited PGP activity by more than 50% compared to the DMSO solvent control (see FIG. 22 B).

In vitro FSP1 activity assays with human (see FIG. 22 C) or murine (see FIG. 22 D) FSP1 were performed as described above. Shown are NADH consumption assays using recombinant, purified human or mouse FSP1 (hFSP1, mFSP1) and resazurine or coenzyme-Q1 (CoQ1) as electron acceptor molecules. After CPBA, comparative compound 2C showed the most inhibition of hFSP1, whereas compounds 2A, 2B, 2C and 2D were effective in inhibiting mFSP1.

Next, we tested if comparative compound 2A (100 μM) can sensitize the two murine hepatocellular carcinoma cell line Hepa 1-6 or Hepa 1c1c7 to FIN56-induced ferroptosis as described above (see FIG. 22 E). Cell viability was assessed using Aquabluer after 16 h. Compound 2A had a small sensitizing effect in Hepa1-6 cells but the effect was not seen in Hepa 1c1c7 cells.

Note that none of the investigated CPBA analogs in series 1 and 2 show superior FSP1-inhibitory activity.

1. A compound according to formula I 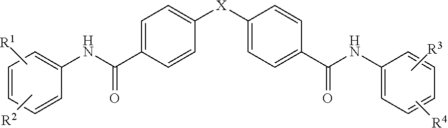
or a salt, tautomer, stereoisomer or N-oxide thereof,
wherein
X is O, S or CH₂;
R¹, R³ are independently of each other selected from H, halogen, CN, NO₂, C₁-C₄-alkyl, C₁-C₄-haloalkyl, C₂-C₆-alkenyl, C₂-C₆-alkynyl, OR^{a}, SR^{a}, C(═O)OR^{a}, NR^{b}R^{c}, and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
R², R⁴ are independently of each other selected from H, halogen, CN, NO₂, C₁-C₄-alkyl, C₁-C₄-haloalkyl, C₂-C₆-alkenyl, C₂-C₆-alkynyl, OR^{a}, SR^{a}, NR^{b}R^{c} and a 3- to 10-membered, saturated, partially or fully unsaturated or aromatic carbocyclyl, heterocyclyl, carbocyclyl-C₁-C₄-alkyl, or heterocyclyl-C₁-C₄-alkyl, wherein said heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{x};
and wherein
R^{a} is H, C₁-C₄-alkyl, C₁-C₄-haloalkyl, or C₂-C₆-alkenyl;
R^{b}, R^{c} are independently of each other selected from H, C₁-C₄-alkyl, and C₂-C₆-alkenyl;
R^{x} is halogen, CN, NO₂, C₁-C₄-alkyl, or C₁-C₄-haloalkyl,
for use in the treatment of cancer.

2. The compound for use according to claim 1, wherein in the compound of formula I
X is O;
R¹, R³ are independently of each other selected from H, halogen and C(═O)OR^{a};
R², R⁴ are independently of each other selected from H, halogen, CN, NO₂, and C₁-C₄-alkyl;
R^{a} is H, or C₁-C₄-alkyl.

3. The compound for use according to claim 1 or 2, wherein in the compound of formula I
X is O;
R¹, R³ are C(═O)OR^{a};
R², R⁴ are H,
R^{a} is H, or C₁-C₄-alkyl.

4. The compound for use according to any one of claims 1 to 3, wherein the compound of formula I is a compound according to the following formula 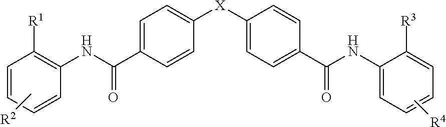
or a pharmaceutically acceptable salt thereof
wherein
X is O;
R¹, R³ are C(═O)OR^{a};
R², R⁴ are H,
R^{a} is H, or C₁-C₄-alkyl.

5. The compound for use according to any one of claims 1 to 4, wherein the compound of formula I is a compound according to the following formula 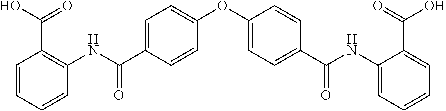 [SMILES: O=C(Nc1ccccc1C(=O)O)c1ccc(Oc2ccc(C(=O)Nc3ccccc3C(=O)O)cc2)cc1]
or a pharmaceutically acceptable salt thereof.

6. The compound for use according to any one of claims 1 to 5, wherein the cancer is in a mammal, preferably in a human and/or a rodent.

7. The compound for use according to any one of claims 1 to 6, wherein the cancer is a mesenchymal cancer and/or in a metastasis prone state.

8. The compound for use according to any one of claims 1 to 7, wherein the cancer is a ferroptosis-sensitive cancer.

9. The compound for use according to any one of claims 1 to 8, wherein the cancer is melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

10. A pharmaceutical composition comprising the compound according to any one of claims 1 to 5 together with a pharmaceutically acceptable carrier and optionally further therapeutic excipient for use in the treatment of cancer, preferably wherein the further therapeutic excipient is a chemotherapeutic agent.

11. The pharmaceutical composition according to claim 10, for use in the treatment of melanoma, breast cancer, head and neck cancer; cancers of the lympho-hematopoietic system including acute myeloid leukemia, (multiple) myeloma and lymphoma, including diffuse large B cell lymphoma; pancreatic cancer, including pancreatic ductal adenocarcinoma; ovarian cancer; cervical cancer; bone cancer, including osteosarcoma; prostate cancer, including prostate adenocarcinoma; kidney cancer, including renal cell carcinoma; lung cancer, including non-small cell lung cancer; liver cancer, including hepatocellular carcinoma and cholangiocarcinoma; gastrointestinal cancer, including colorectal cancer; brain cancer, including glioblastoma; hormone-producing cancer, including adrenocortical carcinoma; or soft tissue cancer including fibrosarcoma.

12. Use of a compound or a pharmaceutically acceptable salt thereof as defined in any one of claims 1 to 5 for inducing phospholipid dependent ferroptosis in an in vitro cell sample.

13. The use according to claim 12, wherein FSP1 function is modulated.

14. The use according to claim 13, wherein the compound as defined in any one of claims 1 to 5 acts as an antagonist of FSP1.

15. The use according to claims 12 to 14, wherein the cell sample is a tumor cell line, an organ slice, organoid, spheroid, assembloid.
